(19) United States
(12) Patent Application Publication
(10) Pub. No.: US 2003/0215442 A1
(43) Pub. Date: Nov. 20, 2003

(54) Methods and compositions for the treatment or prevention of immune disorders using combination therapy

(76) Inventors: Christopher C. Fraser, Lexington, MA (US); Wayne Hancock, Philadelphia, PA (US)

Correspondence Address: Pennie & Edmonds LLP, 1155 Avenue of the Americas, New York, NY, 10036-2711, US

(21) Appl. No.: 10/369,300

(22) Filed: Feb. 19, 2003

Related U.S. Application Data
(60) Provisional application No. 60/358,463, filed on Feb. 19, 2002.

(51) Int. Cl. A61K 48/00; A61K 38/13; A61K 38/17; A61K 31/573; A61K 31/4745; A61K 39/395; A61K 31/522

(52) U.S. Cl. 424/131.1; 514/44; 514/12; 514/11; 514/171; 514/263.32; 514/291

(57) ABSTRACT

The present invention provides methods of suppressing immune disorders such as allograft rejection in a patient by a combination of inhibition of the binding of LIGHT to HVEM and immunosuppressive therapy. Various therapeutic regimens are provided. The present invention further provides kits and pharmaceutical compositions useful in the present methods.

This application claims priority of U.S. Provisional Application No. 60/358,463, filed Feb. 19, 2002, which is incorporated by reference herein in its entirety.

### 1. FIELD OF THE INVENTION

The present invention relates to methods of treating or preventing immune disorders in a patient by combinatorial therapy methods. Such methods entail administering to a patient a combination of (1) a molecule that inhibits binding of LIGHT, a TNF-family ligand, to its receptor, the herpes virus entry mediator (“HVEM”); and (2) an immunosuppressive agent such as cyclosporine.

### 2. BACKGROUND OF THE INVENTION

By binding to structurally related but distinct receptors, tumor necrosis factor (TNF) cytokine family members play key roles in inflammation, immunity and homeostasis (Smith et al., 1994, Cell 76:959-62). An extensive literature exists on how antigenic stimulation of T cells in the absence of costimulation induces anergy, and addition of a second or costimulatory signal such as ligation of CD28 by B7-1 (CD80) or B7-2 (CD86) on dendritic cells promotes a primary T cell response. Various CD28-independent costimulatory pathways are also now recognized. In particular, accruing data suggest that TNF superfamily members can function as costimulatory molecules in T cell activation by delivering a second signal for T cell proliferation, cytokine production and Th1/Th2 differentiation (Locksley et al., 2001, Cell 104:487-501).

LIGHT is a cytokine that is homologous to lymphotoxins, exhibits inducible expression, and competes with herpes simplex virus (HSV) glycoprotein D for herpesvirus entry mediator (HVEM/TR2), a receptor expressed by T lymphocytes (Montgomery et al., 1996, Cell 87:427-36), is a recently identified member of the human (Mauri et al., 1998, Immunity 8:21-30) and mouse (Tamada et al., 2000, Nat. Med. 6:283-9) TNF superfamily. LIGHT is a 29 kDa type II transmembrane protein produced by activated T cells (Mauri et al., 1998, Immunity 8:21-30), as well as monocytes and granulocytes (Zhai et al., 1998, J Clin Invest 102:1142-51), and immature dendritic cells (DC) (Tamada et al., 2000, J. Immunol. 164:4105-10). Apart from its receptor on T cells, HVEM, LIGHT binds to the lymphotoxin β receptor (LTPR) on stromal cells (Mauri et al., 1998, Immunity 8:21-30), and the DcR3/TR6 soluble protein (Yu et al., 1999, J Biol Chem 274:13733-6).

In vitro, LIGHT expression induces potent CD28-independent co-stimulatory activity, leading to NF-KB activation, production of IFN-γ and other cytokines, and T cell proliferation in response to allogeneic DC (Tamada et al., 2000, J. Immunol. 164:4105-10). In vivo studies show that LIGHT promotes cytolytic T cell responses to tumors (Tamada et al., 2000, Nat. Med. 6:283-9; Zhai et al., J Clin Invest 102:1142-51), as well as the development of graft versus host disease (GVHD) (Tamada et al., 2000, Nat. Med. 6:283-9), though no data are yet available concerning the role of the LIGHT/HVEM pathway in solid organ transplantation. The present invention arises from the data presented herein which shows that allograft rejection is repressed by inhibiting the HVEM-LIGHT interaction (by administering a molecule that inhibits the interaction or by disruption of the LIGHT gene) and administering an immunosuppressive agent to a graft recipient.

### 3. SUMMARY OF THE INVENTION

The present invention provides methods and compositions useful to treat or prevent immune system disorders such as allograft rejection. The present invention is based on the discovery that the administration of a combination of (a) a molecule that inhibits the HVEM-LIGHT interaction (referred to herein as an “HVEM-LIGHT inhibitor”) and (b) an immunosuppressive agent results in potentiating administration of either agent alone, unlike what had been observed for administration of combinations of other immunosuppressive agents, such as the combination of anti-CD154 and cyclosporin A.

Accordingly, the present invention provides methods of treating or preventing an immune disorder in a patient, comprising administering to the patient (i) an HVEM-LIGHT inhibitor; and (ii) an immunosuppressive agent, in amounts effective for treating or preventing the immune disorder. The HVEM-LIGHT inhibitor and the immunosuppressive agent can be administered concurrently or successively. Preferably, the HVEM-LIGHT inhibitor and the immunosuppressive agent are administered in amounts that are synergistic.

The present invention provides pharmaceutical compositions comprising (1) an HVEM-LIGHT inhibitor; (2) an immunosuppressive agent; and (3) a pharmaceutically acceptable carrier, wherein the HVEM-LIGHT inhibitor and the immunosuppressive agent are present in amounts effective to prevent or treat the immune disorder.

The present invention further provides kits comprising in one or more containers (1) an HVEM-LIGHT inhibitor; (2) an immunosuppressive agent; (3) and instructions for the use of the HVEM-LIGHT inhibitor and the immunosuppressive agent in the prevention or treatment of immune disorder.

In the foregoing methods and compositions, preferred HVEM-LIGHT inhibitors are soluble HVEM or LIGHT proteins.

In other preferred embodiments, the HVEM-LIGHT inhibitor is an anti-HVEM antibody or an anti-LIGHT antibody.

In yet other preferred embodiments, the HVEM-LIGHT inhibitor is not one or more of the following: a herpesvirus gD protein, a lymphotoxin-α protein, a lymphotoxin-β receptor protein, or a TR6 receptor protein.

In a specific embodiment of the foregoing methods and compositions, for example where the immunosuppressive agent is a drug, the HVEM-LIGHT inhibitor can be conjugated to the immunosuppressive agent.

Immune disorders for which the present methods are useful for treating or preventing include autoimmune diseases, hypersensitivity, and allergic reactions. In a preferred embodiment, the immune disorder is allograft rejection.

The present invention further provides isolated mHVEM nucleic acid molecules, wherein the nucleic acid molecules are selected from the group consisting of: (a) a nucleic acid molecule having a nucleotide sequence which is at least 90% identical to the nucleotide sequence of SEQ ID NO:3 or a complement thereof as determined using the BLAST algorithm; (b) a nucleic acid molecule comprising at least 15 nucleotide residues and having a nucleotide sequence identical to at least 15 consecutive nucleotide residues of residues 1-424 of SEQ ID NO:3 or a complement thereof; (c) a nucleic acid molecule which encodes a polypeptide comprising the amino acid sequence of SEQ ID NO:4; (d) a nucleic acid molecule which encodes a fragment of a polypeptide comprising the amino acid sequence of any of SEQ ID NO:4, wherein the fragment comprises at least 12 consecutive amino acid residues of residues 1-80 of SEQ ID NO:4; and (e) a nucleic acid molecule which encodes a naturally occurring allelic variant of a polypeptide comprising the amino acid sequence of SEQ ID NO:4, wherein the nucleic acid molecule hybridizes under stringent conditions with a nucleic acid molecule consisting of the nucleotide sequence of any of residues 1-424 of SEQ ID NO:3 or a complement thereof.

The present invention further provides isolated mHVEM polypeptides, wherein the polypeptides are selected from the group consisting of: (a) a fragment of a polypeptide comprising the amino acid sequence of SEQ ID NO:4; wherein the fragment comprises at least 12 consecutive amino acid residues of residues 1-80 of SEQ ID NO:4; (b) a naturally occurring allelic variant of a polypeptide comprising the amino acid sequence of any of SEQ ID NO:4, wherein the polypeptide is encoded by a nucleic acid molecule which hybridizes with a nucleic acid molecule consisting of the nucleotide sequence of residues 1-424 of SEQ ID NO:3 or a complement thereof under stringent conditions; and (c) a polypeptide which is encoded by a nucleic acid molecule comprising a nucleotide sequence which is at least 90% identical to a nucleic acid consisting of residues 1-424 of SEQ ID NO:3 or a complement thereof as determined using the BLAST algorithm.

Host cells comprising the mHVEM nucleic acids of the invention are further provided. The present invention also encompasses method of making the mHVEM polypeptides of the invention comprising culturing host cells that contain the nucleic acids of the invention under conditions such that the mHVEM polypeptides are expressed. Yet further, the present invention further encompasses antibodies that bind to the mHVEM polypeptides of the invention but not to human HVEM polypeptides.

### 4. BRIEF DESCRIPTION OF THE FIGURE

FIG. 1: Sequence alignment between the murine and human HVEM proteins. The sequences share 46% sequence identify and 57% sequence similarity. mHVEM=murine HVEM; hHVEM=human HVEM.

### 5. DETAILED DESCRIPTION OF THE INVENTION

The present invention provides methods and compositions useful to treat or prevent allograft rejection. The methods and compositions of the invention are based on the unexpected benefits observed when a combination of an (i) an HVEM-LIGHT inhibitor; and (ii) an immunosuppressive agent is administered to an allograft recipient. Thus, where prevention or treatment of allograft rejection is desired, the methods of the invention entail administering, concurrently or successively, to a patient, prior or following receipt of a transplant, a HVEM-LIGHT inhibitor and the immunosuppressive agent. Such methods are described in more detail in Section 5.1, infra.

The present invention provides pharmaceutical compositions and kits that are useful for practicing the methods of the invention. Such pharmaceutical compositions and kits are described in Sections 5.5 and 5.6 below, respectively.

5.1 Methods of the Invention

Described below are combinatorial methods and related compositions for treating or preventing immune disorders such as allograft rejection. The methods of the invention involve the administration of at least two agents to a patient, the first of which disrupts the interaction between the HVEM receptor and its ligand, LIGHT, and the second of which is an immunosuppressive agent. The agent which disrupts the interaction between HVEM and LIGHT is referred to herein as an HVEM-LIGHT inhibitor. An HVEM-LIGHT inhibitor preferably disrupts the interaction between the HVEM receptor and its ligand LIGHT by at least 20%, more preferably by at least 30%, more preferably by at least 40%, yet more preferably by at least 50%. In certain embodiments, an HVEM-LIGHT inhibitor disrupts the interaction between HVEM and LIGHT by up to 60%, 70%, 80%, or 90%. As used herein, percentage inhibition of the HVEM-LIGHT interaction is measured according to an embodiment of the heterogenous assay described in Section 5.5, infra. Briefly, a protein (such as a fusion protein) comprising a LIGHT-binding portion of HVEM (or an HVEM-binding portion of LIGHT) is immobilized on a solid surface, and contacted with a protein comprising an HVEM-binding portion of LIGHT (or a LIGHT-binding portion of HVEM) in the presence and absence of the test compound. After the reaction is complete, unreacted components are removed (e.g., by washing) and any complexes formed will remain immobilized on the solid surface. A radioactively labeled antibody that binds to the HVEM-binding portion of LIGHT (or to the LIGHT-binding portion of HVEM), but not to the test compound, can be added to the system and allowed to bind to the complexed components. The interaction between HVEM and LIGHT can be detected by measuring the amount of radioactivity that remains associated with the HVEM-LIGHT complex. A successful inhibition of the interaction by the test compound will result in a decrease in measured radioactivity. The % inhibition of the HVEM-LIGHT interaction is the percentage difference in bound radioactivity in the present and absence of test compound; for example, if the amount of bound radioactivity in the presence of the test compound is 70% of bound radioactivity in the absence of the test compound, the test compound is said to inhibit the HVEM-LIGHT interaction by 30%.

The HVEM-LIGHT inhibitor can be a competitive or non-competitive inhibitor of the HVEM-LIGHT interaction. As described in Section 5.2, infra, the HVEM-LIGHT inhibitor can be a protein. In one embodiment, the HVEM-LIGHT inhibitor is a membrane-bound form of LIGHT or HVEM, for example LIGHT or HVEM recombinantly expressed on a cell. For example, an HVEM-expressing cell that does not contain the machinery for mediating the LIGHT signal can be used to inhibit the endogenous HVEM-LIGHT interaction. In more preferred embodiments, the HVEM-LIGHT inhibitor is a soluble protein. In one embodiment, the HVEM-LIGHT inhibitor is a soluble form of HVEM or a soluble form of another receptor to which LIGHT binds. In another embodiment, the HVEM-LIGHT inhibitor is a soluble LIGHT protein or another ligand which binds to HVEM. In yet other embodiments, the HVEM-LIGHT inhibitor is an anti-HVEM or anti-LIGHT antibody. Alternatively, the HVEM-LIGHT inhibitor can be small organic or inorganic molecule of preferably less than 500 daltons in size.

The combinatorial therapy methods of the present invention often result in a greater than additive effect, providing therapeutic benefits where neither the HVEM-LIGHT inhibitor or immunosuppressive agent alone is effective. The outcome of the present therapeutic and prophylactic methods is to at least produce in a patient a healthful benefit, which includes but is not limited to: prolonging the lifespan of a patient, prolonging the onset of symptoms of the disorder, and/or alleviating a symptom of the disorder after onset of a symptom of the disorder. For example, in the context of allograft rejection, the present therapeutic and prophylactic methods can result in prolonging the lifespan of an allograft recipient, prolonging the duration of allograft tolerance prior to rejection, and/or alleviating a symptom associated with allograft rejection.

As used herein, the term “prevention” refers to administration of one or both components of the combinatorial regimens of the invention to the patient before the onset of symptoms or molecular indications of the immune disorder of interest. In contrast, the term “treatment” refers to administration of one or both components of the combinatorial regimens of the present invention to the patient after the onset of symptoms or molecular indications of the disorder. With respect to allograft rejection or graft versus host disease, in a preferred embodiment of the invention, the prophylactic methods of the invention are initiated prior to transplantation of the allograft.

The patients on whom the combinatorial therapy methods of the invention are practiced include, but are not limited to, animals such as cows, pigs, horses, chickens, cats, dogs, etc., and are preferably mammals, and most preferably human.

In the present methods, the HVEM-LIGHT inhibitor and the immunosuppressive agent can be administered concurrently or successively. As used herein, the HVEM-LIGHT inhibitor and the immunosuppressive agent are said to be administered concurrently if they are administered to the patient on the same day, for example, simultaneously, or 1, 2, 3, 4, 5, 6, 7 or 8 hours apart. In contrast, the HVEM-LIGHT inhibitor and the immunosuppressive agent are said to be administered successively if they are administered to the patient on the different days, for example, the HVEM-LIGHT inhibitor and the immunosuppressive agent can be administered at a 1-day, 2-day or 3-day intervals. In the methods of the present invention, administration of the HVEM-LIGHT inhibitor can precede or follow administration of the immunosuppressive agent.

The therapeutic and prophylactic regimens of the present invention can include both concurrent and successive administration. As a non-limiting example, the HVEM-LIGHT inhibitor and immunosuppressive agent can be administered concurrently for a period of time, followed by a second period of time in which the administration of the HVEM-LIGHT inhibitor and the immunosuppressive agent is alternated.

The therapeutic regimens of the present invention can be practiced as long as the treatment or prevention of an immune disorder is required or desired.

Because of the synergistic effects of administering a HVEM-LIGHT inhibitor and an immunosuppressive agent, such agents can be administered in amounts that, if one or both of the agents is administered alone, is/are not effective for treating an immune disorder of interest.

### 5.1.1 Allograft Rejection and Graft Versus Host Disease

In preferred embodiments of the present invention, the methods and compositions of the present invention are used to treat or prevent allograft rejection or graft versus host disease.

The methods and compositions of the invention can be used in the prevention or treatment of any type of allograft rejection or graft versus host disease for any type of graft, including a xenograft. The allograft can be an organ transplant, such as, but not limited to, a heart, kidney, liver, lung or pancreas. Alternatively, the allograft can be a tissue transplant, such as, but not limited to, heart valve, endothelial, cornea, eye lens or bone marrow tissue transplant. In yet other embodiments, the allograft can be a skin graft.

In certain embodiments, the present methods can be practiced for a day, three days, a week, two weeks or a month prior to a transplantation. In other embodiments, the present methods can be practiced for a week, two weeks, three weeks, one month, two months, three months or six months following a transplantation. In a preferred embodiment, the present methods are practiced both before and after a transplantation is carried out.

### 5.1.2 Disorders of the Invention

The methods and compositions of the present invention are useful for treating or preventing a variety of immune disorders, including but not limited to autoimmune diseases, hypersensitivity, and allergic reactions. Such disorders are referred to herein as disorders of the invention.

Exemplary disorders of the invention include allograft rejection, delayed-type hypersensitivity, graft versus host disease, drug allergies, atrophic gastrititis, thyroiditis, allergic encephalomyelitis, autoimmune hemolytic anemia, sympathetic ophthalmia, systemic lupus erythematosus (“SLE”), rheumatoid arthritis, multiple sclerosis, asthma, inflammatory bowel disease (“IBD”), and myasthenia gravis.

5.2 HVEM-LIGHT Inhibitors

As mentioned in Section 5.1, supra, many types of molecules can be used as HVEM-LIGHT inhibitors. Such molecules include polypeptides, peptides, antibodies, and small molecules. Below is a description of exemplary HVEM-LIGHT inhibitors. In certain specific embodiments of the invention, the HVEM-LIGHT inhibitor employed in the present methods and compositions is not one or more of the following: a herpesvirus gD protein, a lymphotoxin-a protein, a lymphotoxin-β receptor protein, or a TR6 receptor protein.

### 5.2.1 Dominant Negative Forms of LIGHT- and HVEM-Binding Polypeptides

The present invention encompasses the use of dominant negative forms of polypeptides that bind to the HVEM or LIGHT proteins in the methods and compositions of the present invention. Such dominant negative proteins include full length HVEM proteins (for example, expressed by a cell that is administered to a patient) or peptide fragments thereof which bind to the LIGHT ligand, as well as full length LIGHT proteins (for example, expressed by a cell that is administered to a patient) or peptide fragments thereof which bind to the HVEM receptor.

Also included are dominant negative forms of HVEM ligands other than LIGHT (e.g., herpesvirus gD protein (Genbank accession no. L09242; SEQ ID NO: 13), lymphotoxin-α Genbank accession no. AY070490; SEQ ID NO: 15), as well as dominant negative forms of receptors to which LIGHT binds other than HVEM (e.g., the lymphotoxin-β receptor (Genbank accession no. XM_{—}033305; SEQ ID NO:17), the TR6 receptor (Genbank accession no. AF134240; SEQ ID NO: 19; see also Yu et al., 1999, J. Biol. Chem. 274:13733-36)).

The amino acid sequences depicted in SEQ ID NO:2 (see WO 00/14230, where HVEM is referred to as TANGO-69-receptor) and SEQ ID NO:4 represent full length human and murine HVEM proteins, respectively. The amino acid sequences depicted in SEQ ID NO:6 (see Genbank accession no. AF036581) and SEQ ID NO:8 (see WO 99/11662, where LIGHT is referred to as TANGO-69) represent full length human and murine LIGHT proteins, respectively.

Preferred in the present methods ane compositions are soluble LIGHT and HVEM polypeptides. Such polypeptides generally lack a transmembrane domain and an intracellular domain.

After HVEM is processed into a mature protein, the extracellular domain of human HVEM consists of amino acids 39-201 of the full protein (SEQ ID NO:2) (Whitbeck et al., 2001, J. Virol. 75:171-80). The use of the entire HVEM extracellular domain, or a LIGHT binding portion thereof, is contemplated in the present methods an compositions. WO 00/14230 teaches that there are three known soluble forms of the normally membrane-bound HVEM, referred to sHVEM1 (SEQ ID NO:9), sHVEM2 (SEQ ID NO:10), and sHVEM3 (SEQ ID NO:11). The use of these soluble forms, or a LIGHT-binding portion thereof, is also desirable in the present methods.

Fragments of HVEM or LIGHT that are useful in the methods and compositions present invention may contain deletions, additions or substitutions of amino acid residues within the amino acid sequence encoded by an HVEM or LIGHT gene. Preferably mutations result in a silent change, thus producing a functionally equivalent HVEM or LIGHT gene product.

An HVEM or LIGHT polypeptide sequence preferably comprises an amino acid sequence that exhibits at least about 65% sequence similarity to human HVEM or LIGHT, more preferably exhibits at least 70% sequence similarity to human HVEM or LIGHT, yet more preferably exhibits at least about 75% sequence similarity human HVEM or LIGHT. In other embodiments, the HVEM or LIGHT polypeptide sequence preferably comprises an amino acid sequence that exhibits at least 85% sequence similarity to human HVEM or LIGHT, yet more preferably exhibits at least 90% sequence similarity to human HVEM or LIGHT, and most preferably exhibits at least about 95% sequence similarity to human HVEM or LIGHT. In one embodiment, such a polypeptide sequence comprises all or a portion of the murine HVEM or LIGHT sequence, respectively.

In other embodiment, the HVEM or LIGHT polypeptide sequence preferably comprises an amino acid sequence that exhibits at least about 65% sequence identity to murine HVEM or LIGHT, more preferably exhibits at least 70% sequence identity to murine HVEM or LIGHT, yet more preferably exhibits at least about 75% sequence identity to murine HVEM or LIGHT. In other embodiments, the HVEM or LIGHT polypeptide sequence preferably comprises an amino acid sequence that exhibits at least 85% sequence identity to murine HVEM or LIGHT, yet more preferably exhibits at least 90% sequence identity to murine HVEM or LIGHT, and most preferably exhibits at least about 95% sequence identity to murine HVEM or LIGHT. In one embodiment, such a polypeptide sequence comprises a portion of murine HVEM that binds to the human LIGHT extracellular domain, or a portion of murine LIGHT that binds to the human HVEM extracellular domain, respectively.

Additional polypeptides suitable in the present methods are those encoded by the nucleic acids described in Section 5.4.1 below.

The determination of percent identity between two sequences can be accomplished using a mathematical algorithm. A preferred, non-limiting example of a mathematical algorithm utilized for the comparison of two sequences is the algorithm of Karlin and Altschul (1990) Proc Natl Acad Sci. 87:2264-2268, modified as in Karlin and Altschul (1993) Proc Natl Acad Sci. 90:5873-5877. Such an algorithm is incorporated into the NBLAST and XBLAST programs of Altschul et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used.

Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (1994) Comput. Appl. Biosci., 10:3-5; and FASTA described in Pearson and Lipman (1988) 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. For a further description of FASTA parameters, see http://bioweb.pasteur.fr/docs/man/man/fasta.1.html#sect2.

The percent identity between two sequences can be determined using techniques similar to those described above, with or without allowing gaps. In calculating percent identity, only exact matches are counted. However, conservative substitutions should be considered in evaluating sequences that have a low percent identity with the HVEM or LIGHT sequences disclosed herein.

In a specific embodiment, polypeptides comprising at least 10, 20, 30, 40, 50, 75, 100, or 200 amino acids of SEQ ID NO:2 or 4 that bind to LIGHT, or polypeptides comprising at least 10, 20, 30, 40, 50, 75, 100, or 200 amino acids of SEQ ID NO:6 or 8 that bind to HVEM, are used in the present invention. In a preferred embodiment, such a polypeptide comprises all or a portion of the extracellular domain of SEQ ID NO:2, 4, 6, or 8.

### 5.2.1.1 Fusion Proteins

Also useful in the present methods and compositions also are fusion proteins comprising a portion of an HVEM-binding ligand or a LIGHT-binding receptor polypeptide sequence which binds to HVEM or LIGHT, respectively, operatively associated to a heterologous component, e.g., a heterologous peptide. Heterologous components can include, but are not limited to sequences which facilitate isolation and purification of the fusion protein. Heterologous components can also include sequences which confer stability to the LIGHT- or HVEM-binding polypeptides. Such fusion partners are well known to those of skill in the art.

The present invention encompasses the use of fusion proteins comprising an HVEM (e.g., SEQ ID NO:2 or SEQ ID NO:4) or LIGHT polypeptide (SEQ ID NO:6 and SEQ ID NO:8) and a heterologous polypeptide (i.e., an unrelated polypeptide or fragment thereof, preferably at least 10, at least 20, at least 30, at least 40, at least 50, at least 60, at least 70, at least 80, at least 90 or at least 100 amino acids of the polypeptide). The present invention encompasses the use of fusion proteins comprising an herpesvirus gD protein (SEQ ID NO:13), lymphotoxin-α (SEQ ID NO:15), the lymphotoxin-β receptor (SEQ ID NO: 17), or the TR6 receptor (SEQ ID NO:19) and a heterologous polypeptide (i.e., an unrelated polypeptide or fragment thereof, preferably at least 10, at least 20, at least 30, at least 40, at least 50, at least 60, at least 70, at least 80, at least 90 or at least 100 amino acids of the polypeptide). The fusion can be direct, but may occur through linker sequences. The heterologous polypeptide may be fused to the N-terminus or C-terminus of an LIGHT- or HVEM-binding polypeptide.

A fusion protein can comprise an LIGHT- or HVEM-binding polypeptide fused to a heterologous signal sequence at its N-terminus. Various signal sequences are commercially available. Eukaryotic heterologous signal sequences include, but art not limited to, the secretory sequences of melittin and human placental alkaline phosphatase (Stratagene; La Jolla, Calif.). Prokaryotic heterologous signal sequences useful in the methods of the invention include, but are not limited to, the phoA secretory signal (Sambrook et al, eds., Molecular Cloning: A Laboratory Manual, 2nd ed., Cold Spring Harbor Laboratory, Cold Spring Harbor Laboratory Press, Cold Spring Harbor, N.Y., 1989) and the protein A secretory signal (Pharmacia Biotech; Piscataway, N.J.).

The LIGHT- or HVEM-binding protein or fragment thereof can be fused to tag sequences, e.g., a hexa-histidine peptide, such as the tag provided in a pQE vector (QIAGEN, Inc., Chatsworth, Calif., 91311), among others, many of which are commercially available for use in the methods of the invention. As described in Gentz et al., 1989, Proc. Natl. Acad. Sci. USA, 86:821-824, for instance, hexa-histidine provides for convenient purification of the fusion protein. Other examples of peptide tags are the hemagglutinin “HA” tag, which corresponds to an epitope derived from the influenza hemagglutinin protein (Wilson et al., 1984, Cell, 37:767) and the “flag” tag (Knappik et al., 1994, Biotechniques, 17(4):754-761). These tags are especially useful for purification of recombinantly produced polypeptides of the invention.

Any fusion protein may be readily purified by utilizing an antibody specific or selective for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972). In this system, the gene of interest is subcloned into a vaccinia recombination plasmid such that the open reading frame of the gene is translationally fused to an amino-terminal tag consisting of six histidine residues. Extracts from cells infected with recombinant vaccinia virus are loaded onto Ni²⁺•nitriloacetic acid-agarose columns and histidine-tagged proteins are selectively eluted with imidazole-containing buffers.

An affinity label can also be fused at its amino terminal to the carboxyl terminal of the LIGHT- or HVEM-binding protein or fragment thereof for use in the methods of the invention. The precise site at which the fusion is made in the carboxyl terminal is not critical. The optimal site can be determined by routine experimentation. An affinity label can also be fused at its carboxyl terminal to the amino terminal of the LIGHT- or HVEM-binding polypeptide for use in the methods of the invention.

A variety of affinity labels known in the art may be used, such as, but not limited to, the immunoglobulin constant regions (see also Petty, 1996, Metal-chelate affinity chromatography, in Current Protocols in Molecular Biology, Vol. 2, Ed. Ausubel et al., Greene Publish. Assoc. & Wiley Interscience), glutathione S-transferase (GST; Smith, 1993, Methods Mol. Cell Bio. 4:220-229), the E. coli maltose binding protein (Guan et al., 1987, Gene 67:21-30), and various cellulose binding domains (U.S. Pat. Nos. 5,496,934; 5,202,247; 5,137,819; Tomme et al., 1994, Protein Eng. 7:117-123), etc. Other affinity labels are recognized by specific binding partners and thus facilitate isolation by affinity binding to the binding partner which can be immobilized onto a solid support. Some affinity labels may afford the LIGHT- or HVEM-binding polypeptide novel structural properties, such as the ability to form multimers. These affinity labels are usually derived from proteins that normally exist as homopolymers. Affinity labels such as the extracellular domains of CD8 (Shiue et al., 1988, J. Exp. Med. 168:1993-2005), or CD28 (Lee et al., 1990, J. Immunol. 145:344-352), or fragments of the immunoglobulin molecule containing sites for interchain disulfide bonds, could lead to the formation of multimers.

As will be appreciated by those skilled in the art, many methods can be used to obtain the coding region of the above-mentioned affinity labels, including but not limited to, DNA cloning, DNA amplification, and synthetic methods. Some of the affinity labels and reagents for their detection and isolation are available commercially.

A preferred affinity label is a non-variable portion of the immunoglobulin molecule. Typically, such portions comprise at least a functionally operative CH2 and CH3 domain of the constant region of an immunoglobulin heavy chain. Fusions are also made using the carboxyl terminus of the Fc portion of a constant domain, or a region immediately amino-terminal to the CH1 of the heavy or light chain. Suitable immunoglobulin-based affinity label may be obtained from IgG-1, -2, -3, or -4 subtypes, IgA, IgE, IgD, or IgM, but preferably IgG1. Preferably, a human immunoglobulin is used when the LIGHT- or HVEM-binding polypeptide is intended for in vivo use for humans. Many DNA encoding immunoglobulin light or heavy chain constant regions are known or readily available from cDNA libraries. See, for example, Adams et al., Biochemistry, 1980, 19:2711-2719; Gough et al., 1980, Biochemistry, 19:2702-2710; Dolby et al., 1980, Proc. Natl. Acad. Sci. U.S.A., 77:6027-6031; Rice et al., 1982, Proc. Natl. Acad. Sci. U.S.A., 79:7862-7865; Falkner et al., 1982, Nature, 298:286-288; and Morrison et al., 1984, Ann. Rev. Immunol, 2:239-256. Because many immunological reagents and labeling systems are available for the detection of immunoglobulins, the LIGHT- or HVEM-binding polypeptide-Ig fusion protein can readily be detected and quantified by a variety of immunological techniques known in the art, such as the use of enzyme-linked immunosorbent assay (ELISA), immunoprecipitation, fluorescence activated cell sorting (FACS), etc. Similarly, if the affinity label is an epitope with readily available antibodies, such reagents can be used with the techniques mentioned above to detect, quantitate, and isolate the LIGHT- or HVEM-binding polypeptide containing the affinity label. In many instances, there is no need to develop specific or selective antibodies to the LIGHT- or HVEM-binding polypeptide for the purposes of purification.

A fusion protein can comprise an LIGHT- or HVEM-binding polypeptide fused to the Fc domain of an immunoglobulin molecule or a fragment thereof for use in the methods of the invention. A fusion protein can also comprise an LIGHT- or HVEM-binding polypeptide fused to the CH2 and/or CH3 region of the Fe domain of an immunoglobulin molecule. Furthermore, a fusion protein can comprise an LIGHT- or HVEM-binding polypeptide fused to the CH2, CH3, and hinge regions of the Fe domain of an immunoglobulin molecule (see Bowen et al., 1996, J Immunol. 156:442-49). This hinge region contains three cysteine residues which are normally involved in disulfide bonding with other cysteines in the Ig molecule. Since none of the cysteines are required for the peptide to function as a tag, one or more of these cysteine residues may optionally be substituted by another amino acid residue, such as for example, serine.

Various leader sequences known in the art can be used for the efficient secretion of the LIGHT- or HVEM-binding polypeptide from bacterial and mammalian cells (von Heijne, 1985, J. Mol. Biol. 184:99-105). Leader peptides are selected based on the intended host cell, and may include bacterial, yeast, viral, animal, and mammalian sequences. For example, the herpes virus glycoprotein D leader peptide is suitable for use in a variety of mammalian cells. A preferred leader peptide for use in mammalian cells can be obtained from the V-J2-C region of the mouse immunoglobulin kappa chain (Bernard et al., 1981, Proc. Natl. Acad. Sci. 78:5812-5816). Preferred leader sequences for targeting HVEM or LIGHT polypeptide expression in bacterial cells include, but are not limited to, the leader sequences of the E. coli proteins OmpA (Hobom et al., 1995, Dev. Biol. Stand. 84:255-262), Pho A (Oka et al., 1985, Proc. Natl. Acad. Sci 82:7212-16), OmpT (Johnson et al., 1996, Protein Expression 7:104-113), LamB and OmpF (Hoffman & Wright, 1985, Proc. Natl. Acad. Sci. USA 82:5107-5111), β-lactamase (Kadonaga et al., 1984, J. Biol. Chem. 259:2149-54), enterotoxins (Morioka-Fujimoto et al., 1991, J. Biol. Chem. 266:1728-32), and the Staphylococcus aureus protein A (Abrahmsen et al., 1986, Nucleic Acids Res. 14:7487-7500), and the B. subtilis endoglucanase (Lo et al., Appl. Environ. Microbiol. 54:2287-2292), as well as artificial and synthetic signal sequences (MacIntyre et al., 1990, Mol. Gen. Genet. 221:466-74; Kaiser et al., 1987, Science, 235:312-317).

Fusion proteins can be produced by standard recombinant DNA techniques or by protein synthetic techniques, e.g., by use of a peptide synthesizer. For example, a nucleic acid molecule encoding a fusion protein can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al., eds., John Wiley & Sons, 1992).

The nucleotide sequence coding for a fusion protein can be inserted into an appropriate expression vector, i.e., a vector which contains the necessary elements for the transcription and translation of the inserted protein-coding sequence. The expression of a fusion protein may be regulated by a constitutive, inducible or tissue-specific or -selective promoter. It will be understood by the skilled artisan that fusion proteins, which can facilitate solubility and/or expression, and can increase the in vivo half-life of the LIGHT- or HVEM-binding polypeptide and thus are useful in the methods of the invention. The LIGHT- or HVEM-binding polypeptides or peptide fragments thereof, or fusion proteins can be used in any assay that detects or measures LIGHT- or HVEM-binding polypeptides or in the calibration and standardization of such assay.

The methods of invention encompass the use of LIGHT- or HVEM-binding polypeptides or peptide fragments thereof, which may be produced by recombinant DNA technology using techniques well known in the art. Thus, methods for preparing the LIGHT- or HVEM-binding polypeptides and peptides of the invention by expressing nucleic acid containing LIGHT- or HVEM-binding gene sequences are described herein. Methods which are well known to those skilled in the art can be used to construct expression vectors containing, e.g., HVEM polypeptide coding sequences (including but not limited to nucleic acids encoding all or a LIGHT-binding portion of SEQ ID NO:9, SEQ ID NO:10, or SEQ ID NO:11) or LIGHT polypeptide coding sequences and appropriate transcriptional and translational control signals. These methods include, for example, in vitro recombinant DNA techniques, synthetic techniques, and in vivo genetic recombination. See, for example, the techniques described in Sambrook et al., 1989, supra, and Ausubel et al., 1989, supra. Alternatively, RNA capable of encoding LIGHT- or HVEM-binding polypeptide sequences may be chemically synthesized using, for example, synthesizers (see e.g., the techniques described in Oligonucleotide Synthesis, 1984, Gait, M. J. ed., IRL Press, Oxford).

### 5.2.1.2 Expression Systems

A variety of host-expression vector systems may be utilized to express the LIGHT- or HVEM-binding polypeptide coding sequences for use in the methods of the invention. Such host-expression systems represent vehicles by which the coding sequences of interest may be produced and subsequently purified, but also represent cells which may, when transformed or transfected with the appropriate nucleotide coding sequences, exhibit the LIGHT- or HVEM-binding polypeptide of the invention in situ. These include but are not limited to microorganisms such as bacteria (e.g., E. coli, B. subtilis) transformed with recombinant bacteriophage DNA, plasmid DNA or cosmid DNA expression vectors containing LIGHT- or HVEM-binding polypeptide coding sequences; yeast (e.g., Saccharomyces, Pichia) transformed with recombinant yeast expression vectors containing the LIGHT- or HVEM-binding polypeptide coding sequences; insect cell systems infected with recombinant virus expression vectors (e.g., baculovirus) containing the LIGHT- or HVEM-binding polypeptide coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing LIGHT- or HVEM-binding polypeptide coding sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the LIGHT- or HVEM-binding polypeptide being expressed. For example, when a large quantity of such a protein is to be produced, for the generation of pharmaceutical compositions of LIGHT- or HVEM-binding protein or for raising antibodies to HVEM or LIGHT protein, vectors which direct the expression of high levels of fusion protein products that are readily purified may be desirable. Such vectors include, but are not limited, to the E. coli expression vector pUR278 (Ruther et al., 1983, EMBO J. 2:1791), in which the HVEM or LIGHT polypeptide coding sequence may be ligated individually into the vector in frame with the lac Z coding region so that a fusion protein is produced; pIN vectors (Inouye & Inouye, 1985, Nucleic Acids Res. 13:3101; Van Heeke & Schuster, 1989, J Biol. Chem. 264:5503); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include, e.g., thrombin or factor Xa protease cleavage sites so that the cloned target polypeptide can be released from the GST moiety.

In an insect system, Autographa californica nuclear polyhedrosis virus (AcNPV) is used as a vector to express foreign genes. The virus grows in Spodoptera frugiperda cells. The LIGHT- or HVEM-binding polypeptide coding sequence may be cloned individually into non-essential regions (for example the polyhedrin gene) of the virus and placed under control of an AcNPV promoter (for example the polyhedrin promoter). Successful insertion of LIGHT- or HVEM-binding polypeptide coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (e.g., see Smith et al., 1983, J. Virol. 46:584; Smith, U.S. Pat. No. 4,215,051).

In mammalian host cells, a number of viral-based expression systems may be utilized. In cases where an adenovirus is used as an expression vector, the LIGHT- or HVEM-binding polypeptide coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing HVEM or LIGHT polypeptide in infected hosts. (See, e.g., Logan & Shenk, 1984, Proc. Natl. Acad. Sci. USA 81:3655). Specific initiation signals may also be required for efficient translation of inserted LIGHT- or HVEM-binding polypeptide coding sequences. These signals include the ATG initiation codon and adjacent sequences. In cases where an entire LIGHT- or HVEM-binding polypeptide gene, including its own initiation codon and adjacent sequences, is inserted into the appropriate expression vector, no additional translational control signals may be needed. However, in cases where only a portion of the LIGHT- or HVEM-binding polypeptide coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (See Bittner et al., 1987, Methods in Enzymol. 153:516).

In addition, a host cell strain may be chosen which modulates the expression of the inserted sequences, or modifies and processes the polypeptide in the specific fashion desired. Such modifications (e.g., glycosylation) and processing (e.g., cleavage) of protein products may be important for the function of the protein. Different host cells have characteristic and specific mechanisms for the post-translational processing and modification of proteins and polypeptides. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the polypeptide may be used. Such mammalian host cells include but are not limited to CHO, VERO, BHK, HeLa, COS, MDCK, 293, 3T3, W138.

For long-term, high-yield production of recombinant proteins, stable expression is preferred. For example, cell lines which stably express the LIGHT- or HVEM-binding polypeptide may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the LIGHT- or HVEM-binding polypeptide. Such engineered cell lines may be particularly useful in screening and evaluation of compounds that affect the endogenous activity of the HVEM or LIGHT polypeptide.

A number of selection systems may be used, including but not limited to the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk⁻, hgprt⁻ or aprt⁻ cells, respectively. Also, antimetabolite resistance can be used as the basis of selection for the following genes: dhfr, which confers resistance to methotrexate (Wigler et al., 1980, Proc Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147).

### 5.2.2 Antibodies

The methods of the present invention encompass the use of antibodies or fragments thereof capable of specifically or selectively recognizing one or more HVEM or LIGHT polypeptide epitopes or epitopes of conserved variants or peptide fragments of the HVEM or LIGHT polypeptides. Such antibodies may include, but are not limited to, polyclonal antibodies, monoclonal antibodies (mAbs), humanized or chimeric antibodies, single chain antibodies, Fab fragments, F(ab′)₂ fragments, Fv fragments, fragments produced by a Fab expression library, anti-idiotypic (anti-Id) antibodies, and epitope-binding fragments of any of the above. In a preferred embodiment, the anti-HVEM or anti-LIGHT antibody used in the present methods binds to the HVEM or LIGHT extracellular domain.

Described herein are methods for the production of antibodies or fragments thereof. Any of such antibodies or fragments thereof may be produced by standard immunological methods or by recombinant expression of nucleic acid molecules encoding the antibody or fragments thereof in an appropriate host organism.

For the production of antibodies against an HVEM or LIGHT polypeptide, various host animals may be immunized by injection with an HVEM or LIGHT polypeptide or peptide. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as an HVEM or LIGHT polypeptide, or an antigenic functional derivative thereof. For the production of polyclonal antibodies, host animals such as those described above, may be immunized by injection with HVEM or LIGHT polypeptide supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to, the hybridoma technique of Kohler and Milstein, (1975, Nature 256:495; and U.S. Pat. No. 4,376,110), the human B-cell hybridoma technique (Kosbor et al., 1983, Immunology Today 4:72; Cole et al., 1983, Proc. Natl. Acad. Sci. USA 80:2026), and the EBV-hybridoma technique (Cole et al., 1985, Monoclonal Antibodies And Cancer Therapy, Alan R. Liss, Inc., pp. 77). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

Techniques developed for the production of “chimeric antibodies” (Morrison et al., 1984, Proc. Natl. Acad. Sci., 81, 6851-6855; Neuberger et al., 1984, Nature 312, 604-608; Takeda et al., 1985, Nature 314, 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 5,816,397). The invention thus contemplates chimeric antibodies that are specific or selective for an HVEM or LIGHT polypeptide.

Examples of techniques that have been developed for the production of humanized antibodies are known in the art. (See, e.g., Queen, U.S. Pat. No. 5,585,089 and Winter, U.S. Pat. No. 5,225,539.) An immunoglobulin light or heavy chain variable region consists of a “framework” region interrupted by three hypervariable regions, referred to as complementarity-determining regions (CDRs). The extent of the framework region and CDRs have been precisely defined (see, “Sequences of Proteins of Immunological Interest”, Kabat, E. et al., U.S. Department of Health and Human Services (1983). Briefly, humanized antibodies are antibody molecules from non-human species having one or more CDRs from the non-human species and framework regions from a human immunoglobulin molecule. The invention includes the use of humanized antibodies that are specific or selective for an HVEM or LIGHT polypeptide in the methods and compositions of the invention.

Completely human HVEM or LIGHT antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of an HVEM or LIGHT protein. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected HVEM or LIGHT antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

The methods of the invention encompasses the use of an antibody or derivative thereof comprising a heavy or light chain variable domain, said variable domain comprising (a) a set of three complementarity-determining regions (CDRs), in which said set of CDRs are from a monoclonal antibody to an HVEM or LIGHT polypeptide, most preferably to the HVEM or LIGHT extracellular domain, and (b) a set of four framework regions, in which said set of framework regions differs from the set of framework regions in the monoclonal antibody, and in which said antibody or derivative thereof immunospecifically binds to the HVEM or LIGHT polypeptide. Preferably, the set of framework regions is from a human monoclonal antibody, e.g., a human monoclonal antibody that does not bind the polypeptide encoded for by the HVEM or LIGHT gene sequence.

Phage display technology can be used to increase the affinity of an antibody to an HVEM or LIGHT polypeptide. This technique would be useful in obtaining high affinity antibodies to an HVEM or LIGHT polypeptide that could be used in the combinatorial methods of the invention. The technology, referred to as affinity maturation, employs mutagenesis or CDR walking and re-selection using the HVEM or LIGHT polypeptide antigen to identify antibodies that bind with higher affinity to the antigen when compared with the initial or parental antibody (see, e.g., Glaser et al., 1992, J. Immunology 149:3903). Mutagenizing entire codons rather than single nucleotides results in a semi-randomized repertoire of amino acid mutations. Libraries can be constructed consisting of a pool of variant clones each of which differs by a single amino acid alteration in a single CDR and which contain variants representing each possible amino acid substitution for each CDR residue. Mutants with increased binding affinity for the antigen can be screened by contacting the immobilized mutants with labeled antigen. Any screening method known in the art can be used to identify mutant antibodies with increased avidity to the antigen (e.g., ELISA) (See Wu et al., 1998, Proc Natl. Acad Sci. USA 95:6037; Yelton et al., 1995, J Immunology 155:1994). CDR walking which randomizes the light chain is also possible (See Schier et al., 1996, J. Mol. Bio. 263:551).

Alternatively, techniques described for the production of single chain antibodies (U.S. Pat. No. 4,946,778; Bird, 1988, Science 242:423; Huston et al., 1988, Proc. Natl. Acad. Sci. USA 85:5879; and Ward et al., 1989, Nature 334:544) can be adapted to produce single chain antibodies against HVEM or LIGHT polypeptides. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., 1988, Science 242:1038).

The methods of the invention include using an antibody to an HVEM or LIGHT polypeptide, peptide or other derivative, or analog thereof that is a bispecific antibody (see generally, e.g., Fanger and Drakeman, 1995, Drug News and Perspectives 8:133-137). Such a bispecific antibody is genetically engineered to recognize both (1) an epitope and (2) one of a variety of “trigger” molecules, e.g., Fc receptors on myeloid cells, and CD3 and CD2 on T cells, that have been identified as being able to cause a cytotoxic T-cell to destroy a particular target. Such bispecific antibodies can be prepared either by chemical conjugation, hybridoma, or recombinant molecular biology techniques known to the skilled artisan.

Antibody fragments which recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science 246:1275-1281) to allow rapid and easy identification of monoclonal Fab fragments with the desired specificity.

### 5.2.3 LIGHT or HVEM Antisense Compounds

The present invention encompasses the use of LIGHT and HVEM antisense nucleic acid molecules, i.e., molecules which are complementary to a sense nucleic acid encoding LIGHT and HVEM, respectively, e.g., complementary to the coding strand of a double-stranded cDNA molecule or complementary to an mRNA sequencem as HVEM-LIGHT inhibitors. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame) of HVEM or LIGHT. An antisense nucleic acid molecule can be antisense to all or part of a non-coding region of the coding strand of a nucleotide sequence encoding HVEM or LIGHT. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45 or 50 nucleotides or more in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to an HVEM or LIGHT nucleic acid).

The antisense nucleic acid molecules of the invention are typically administered to a subject or generated in situ such that they hybridize with or bind to cellular mRNA and/or genomic DNA encoding HVEM or LIGHT to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of HVEM or LIGHT antisense nucleic acid molecules includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An HVEM or LIGHT antisense nucleic acid molecule can be an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach, (1988), Nature 334:585-591)) can be used to catalytically cleave HVEM or LIGHT mRNA transcripts to thereby inhibit translation of the HVEM or LIGHT protein encoded by the mRNA. A ribozyme having specificity for an HVEM or LIGHT nucleic acid molecule can be designed based upon the nucleotide sequence of the HVEM or LIGHT cDNAs disclosed herein. For example, a derivative of a Tetrahymena L-19 IVS RNA can be constructed in which the nucleotide sequence of the active site is complementary to the nucleotide sequence to be cleaved in a Cech et al. U.S. Pat. No. 4,987,071; and Cech et al. U.S. Pat. No. 5,116,742. Alternatively, an mRNA encoding a polypeptide of the invention can be used to select a catalytic RNA having a specific ribonuclease activity from a pool of RNA molecules. See, e.g., Bartel and Szostak (1993) Science 261:1411-1418.

The invention also encompasses nucleic acid molecules which form triple helical structures. For example, expression of HVEM or LIGHT can be inhibited by targeting nucleotide sequences complementary to the regulatory region of the gene encoding HVEM or LIGHT, respectively (e.g., the promoter and/or enhancer), to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the antisense nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93: 14670-675.

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996), supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al. (1989) Nucleic Acids Res. 17:5973-88). PNA monomers are then coupled in a stepwise manner to produce a chimeric molecule with a 5′ PNA segment and a 3′ DNA segment (Finn et al. (1996) Nucleic Acids Res. 24(17):3357-63). Alternatively, chimeric molecules can be synthesized with a 5′ DNA segment and a 3′ PNA segment (Peterser et al. (1975) Bioorganic Med. Chem. Lett. 5:1119-11124).

In other embodiments, the HVEM or LIGHT antisense oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, the antisense oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the antisense oligonucleotide may be conjugated to another molecule, e.g., a peptide, hybridization triggered cross-linking agent, transport agent, hybridization-triggered cleavage agent, etc.

5.3 Immunosuppressive Agents

As described herein, the present invention encompasses the use of immunosuppressive agents in combination with an HVEM-LIGHT inhibitor to prevent or treat allograft rejection. Any immunosuppressive agent known to those of skill in the art may be used. Such an immunosuppressive agent can be a drug or other small molecule, or a protein, including but not limited to an antibody. As used herein, the term “immunosuppressive agent” excludes HVEM-LIGHT inhibitors with immunosuppressive activity.

In certain specific embodiments of the invention, the immunosuppressive agent is cyclosporine, FK506, rapamycin, or prednisone.

In other embodiments, the immunosuppressive agent is a steroid, most preferably a corticosteroid.

In other embodiments, the immunosuppressive agent is an antibody, most preferably an anti-T cell or anti-B cell antibody. In one embodiment, the antibody is an anti-CD154 antibody. In another embodiment, the antibody is an anti-CD3 antibody such as OKT3. In yet another embodiment, the antibody is an anti-interleukin-2 receptor antibody. Preparation of immunosuppressive antibodies that are suitable for the claimed methods and compositions can be carried out as described supra in Section 5.2.2.

In yet other embodiments, the immunosuppressive agent is a protein, for example a CTLA4-Ig fusion protein or a CD40-Ig fusion protein.

In yet other embodiments, the immunosuppressive agent is an antiproliferative agent, such as, but not limited to azathiopurine or mycophenolate moefitil.

In yet other embodiments, the immunosuppressive agent is a purine analog. In one embodiment, the purine analog is methotrexate. In another embodiment, the purine analog mercaptopurine.

5.4 Gene Therapy

In certain embodiments of the present invention, administration of an HVEM-LIGHT inhibitor or an immunosuppressive agent in the form of gene therapy is contemplated. Gene therapy refers to therapy performed by the administration to a subject of an expressed or expressible nucleic acid. In this embodiment of the invention, the nucleic acids produce their encoded protein that mediates a therapeutic or prophylactic effect.

### 5.4.1 Nucleic Acids Encoding HVEM- and LIGHT-Binding Polypeptides

The present invention provides nucleic acids encoding dominant negative forms of the HVEM- and LIGHT-binding polypeptides described in Section 5.2.1, supra, for use in expression and gene therapy vectors suitable for production or delivery, respectively, of such polypeptides, to a patient in need thereof.

Nucleic acids useful in the gene therapy methods of the present invention encode the minimal domain of an HVEM protein that interacts with LIGHT, or the minimal domain of a LIGHT protein that interacts with HVEM. Such nucleic acids preferably encode soluble, including secreted, HVEM or LIGHT proteins that interfere with endogenous HVEM-LIGHT interactions in the patients to whom they are administered. In a preferred embodiment, a nucleic acid employed in the present gene therapy-based methods encodes a polypeptide consisting essentially of SEQ ID NO:9, SEQ ID NO:10, or SEQ ID NO:11.

Also included are nucleic acids encoding dominant negative forms of HVEM ligands other than LIGHT (e.g., herpesvirus gD protein (SEQ ID NO: 12), lymphotoxin-α (SEQ ID NO:14), as well as nucleic acids encoding dominant negative forms of receptors to which LIGHT binds other than HVEM (e.g., the lymphotoxin-β receptor (SEQ ID NO:16), the TR6 receptor (SEQ ID NO:18)).

The present invention further encompasses the use of nucleic acids comprising a region of homology to a nucleic acid encoding the HVEM-binding domain of LIGHT, or the LIGHT-binding domain of HVEM, as long as such a nucleic acid encodes a polypeptide that can bind to HVEM or LIGHT, respectively, and interfere with endogenous HVEM-LIGHT interactions in a patient to whom the polypeptide is administered. In various embodiments, the region of homology is characterized by at least 50%, at least 55%, at least 60%, at least 65%, at least 70%, at least 75%, at least 80%, at least 85%, at least 90%, at least 95% or at least 98% identity with nucleotides consisting essentially of the regions in the HVEM or LIGHT open reading frames encoding the extracellular domains of the proteins. Methods of determining sequence homology are described in Section 5.2.1 above.

The invention also encompasses the use of nucleic acids that (1) hybridize under stringent, moderate or low stringency hybridization conditions to a nucleic acid consisting essentially of the regions in the HVEM or LIGHT open reading frames encoding the extracellular domains of the proteins and (2) encode polypeptides which bind to LIGHT or HVEM, respectively. Preferably, such encoded polypeptides do not comprise a transmembrane domain.

By way of example and not limitation, procedures using such conditions of low stringency for regions of hybridization of over 90 nucleotides are as follows (see also Shilo and Weinberg, 1981, Proc. Natl. Acad. Sci. U.S.A. 78,:6789-6792). Filters containing DNA are pretreated for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2X SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and re-exposed to film. Other conditions of low stringency which may be used are well known in the art (e.g., as employed for cross-species hybridizations).

Also, by way of example and not limitation, procedures using such conditions of high stringency for regions of hybridization of over 90 nucleotides are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography.

Other conditions of high stringency which may be used depend on the nature of the nucleic acid (e.g., length, GC content, etc.) and the purpose of the hybridization (detection, amplification, etc.) and are well known in the art. For example, stringent hybridization of a nucleic acid of approximately 15-40 bases to a complementary sequence in the polymerase chain reaction (PCR) is done under the following conditions: a salt concentration of 50 mM KCl, a buffer concentration of 10 mM Tris-HCl, a Mg²⁺ concentration of 1.5 mM, a pH of 7-7.5 and an annealing temperature of 55-60° C.

Selection of appropriate conditions for moderate stringencies is also well known in the art (see, e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, © 1987-1997, Current Protocols, © 1994-1997 John Wiley and Sons, Inc.).

The nucleic acids useful in the present methods may be made by any method known in the art. For example, if the nucleotide sequence of the protein is known, a nucleic acid encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the protein, annealing and ligating of those oligonucleotides, and then amplification of the ligated oligonucleotides by PCR.

Alternatively, a nucleic acid that is useful in the present methods may be generated from nucleic acid from a suitable source. If a clone containing a nucleic acid encoding a particular protein is not available, but the sequence of the protein molecule is known, a nucleic acid encoding the protein may be chemically synthesized or obtained from a suitable source (e.g., a cDNA library such as an antibody cDNA library or a cDNA library generated from, or nucleic acid, preferably poly A+RNA, isolated from, any tissue or cells expressing the protein.

Further, a nucleic acid that is useful in the present methods may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

### 5.4.2 Gene Therapy Methods

Any of the methods for gene therapy available in the art can be used in the methods and compositions of the present invention. Exemplary methods are described below.

For general reviews of the methods of gene therapy, see, Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; Morgan and Anderson, 1993, Ann. Rev. Biochem. 62:191-217; May, 1993, TIBTECH 1, 1(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, the therapeutic comprises nucleic acid sequences encoding an HVEM-LIGHT inhibitor and/or an immunosuppressive agent, said nucleic acid sequences being part of expression vectors that express the HVEM-LIGHT inhibitor and/or immunosuppressive agent in a suitable host. In particular, such nucleic acid sequences have promoters operably linked to the HVEM-LIGHT inhibitor or immunosuppressive agent coding region, said promoter being inducible or constitutive, and, optionally, tissue-specific. In another particular embodiment, nucleic acid molecules are used in which the HVEM-LIGHT inhibitor or immunosuppressive agent coding sequences and any other desired sequences are flanked by regions that promote homologous recombination at a desired site in the genome, thus providing for intrachromosomal expression of the HVEM-LIGHT inhibitor or immunosuppressive agent (Koller and Smithies, 1989, Proc. Natl. Acad. Sci. USA 86:8932-8935; Zijlstra et al., 1989, Nature 342:435-438.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, for example by constructing them as part of an appropriate nucleic acid expression vector and administering the vector so that the nucleic acid sequences become intracellular. Gene therapy vectors can be administered by infection using defective or attenuated retrovirals or other viral vectors (see, e.g., U.S. Pat. No. 4,980,286); direct injection of naked DNA; use of microparticle bombardment (e.g., a gene gun; Biolistic, Dupont); coating with lipids or cell-surface receptors or transfecting agents; encapsulation in liposomes, microparticles, or microcapsules; administration in linkage to a peptide which is known to enter the nucleus; administration in linkage to a ligand subject to receptor-mediated endocytosis (see, e.g., Wu and Wu, 1987, J. Biol. Chem. 262:4429-4432) (which can be used to target cell types specifically expressing the receptors); etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; W092/20316; W093/14188, and WO 93/20221). Alternatively, the nucleic acid can be introduced intracellularly and incorporated within host cell DNA for expression by homologous recombination (Koller and Smithies, 1989, Proc. Natl. Acad. Sci. USA 86:8932-8935; Zijlstra et al., 1989, Nature 342:435-438).

In a specific embodiment, viral vectors that contain nucleic acid sequences encoding an HVEM-LIGHT inhibitor or immunosuppressive agent are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the HVEM-LIGHT inhibitor or immunosuppressive agent to be used in gene therapy are cloned into one or more vectors, thereby facilitating delivery of the gene into a patient. More detail about retroviral vectors can be found in Boesen et al., 1994, Biotherapy 6:29 1-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

### 5.4.2.1 Cell Therapy

One approach to gene therapy encompassed by the present methods and compositions involves transferring a gene, e.g., an HVEM-LIGHT inhibitor or immunosuppressive agent gene, to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to fibroblasts; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences encoding an HVEM-LIGHT inhibitor or immunosuppressive agent are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple and Anderson, 1992, Cell 71:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

5.5 Assays for HVEM-LIGHT Inhibitors

In addition to the LIGHT-HVEM described herein, novel HVEM-LIGHT inhibitors, i.e., agents that interfere with the HVEM-LIGHT interaction, can be identified by the methods described below.

The basic principle of the assay systems used to identify HVEM-LIGHT inhibitors involves preparing a reaction mixture containing at least the HVEM-binding portion of LIGHT and the LIGHT-binding portion of HVEM under conditions (referred to in this section as the LIGHT protein and the HVEM protein, respectively) and for a time sufficient to allow the two to interact and bind, thus forming a complex. In order to test a compound for inhibitory activity, the reaction mixture is prepared in the presence and absence of the test compound. The test compound can be initially included in the reaction mixture, or can be added at a time subsequent to the addition of the HVEM and LIGHT proteins. Control reaction mixtures are incubated without the test compound or with a placebo. The formation of any complexes between the HVEM and LIGHT is then detected. The formation of a complex in the control reaction, but not in the reaction mixture containing the test compound, indicates that the compound interferes with the HVEM-LIGHT interaction.

The assay for compounds that interfere with the interaction of HVEM and LIGHT can be conducted in a heterogeneous or homogeneous format. Heterogeneous assays involve anchoring either the HVEM or LIGHT protein onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between LIGHT and HVEM, e.g., by competition, can be identified by conducting the reaction in the presence of the test substance; i.e., by adding the test substance to the reaction mixture prior to or simultaneously with the HVEM and LIGHT proteins. Alternatively, test compounds that disrupt preformed complexes, e.g. compounds with higher binding constants that displace one of the components from the complex, can be tested by adding the test compound to the reaction mixture after complexes have been formed. The various formats are described briefly below.

In a heterogeneous assay system, either the HVEM or LIGHT protein, is anchored onto a solid surface, while the non-anchored species is labeled, either directly or indirectly. In practice, microtiter plates are conveniently utilized. The anchored species can be immobilized by non-covalent or covalent attachments. Non-covalent attachment can be accomplished simply by coating the solid surface with a solution of the HVEM or LIGHT protein and drying. Alternatively, an immobilized antibody specific for the species to be anchored can be used to anchor the species to the solid surface. The surfaces can be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of the HVEM and LIGHT proteins is prepared in which either the HVEM or LIGHT protein is labeled, but the signal generated by the label is quenched due to complex formation (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances which disrupt HVEM-LIGHT interaction can be identified.

In a particular embodiment, the target gene product can be prepared for immobilization using recombinant DNA techniques known to those of skill in the art. For example, the HVEM or LIGHT protein can be fused to a glutathione-S-transferase (GST) gene using a fusion vector, such as pGEX-5X-1, in such a manner that its binding activity is maintained in the resulting fusion protein. The binding partner (i.e., the LIGHT or HVEM protein, respectively) can be purified and used to raise a monoclonal antibody, using methods routinely practiced in the art and described above. This antibody can be labeled with the radioactive isotope ¹²⁵I, for example, by methods routinely practiced in the art. In a heterogeneous assay, e.g., the GST-LIGHT or GST-HVEM fusion protein can be anchored to glutathione-agarose beads. The HVEM or LIGHT protein, respectively, can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between HVEM and LIGHT can be detected by measuring the amount of radioactivity that remains associated with the glutathione-agarose beads. A successful inhibition of the interaction by the test compound will result in a decrease in measured radioactivity.

Alternatively, the GST-HVEM or GST-LIGHT fusion protein and its binding partner (i.e., LIGHT or HVEM protein, respectively) can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the HVEM-LIGHT interaction can be detected by adding the labeled antibody and measuring the radioactivity associated with the beads.

In one embodiment of the foregoing methods, the test compound is a peptide fragment that corresponds to the extracellular portion of HVEM or LIGHT, thereby allowing the identification of small HVEM-LIGHT inhibitor peptides that can be produced synthetically instead of recombinantly for use in the present methods and compositions.

### 5.5.1 Cell and Animal-Based Model Systems

Described herein are cell- and animal-based systems which act as models for immune disorders of the present invention. Such model systems can be used to test compounds identified, e.g., using the in vitro assays described in Section 5.4.1, above, for their ability and/or effectiveness in treating (e.g., ameliorating or modulating symptoms of) immune-related disorders. Thus, the animal- and cell-based models of the invention can be used to identify drugs, pharmaceuticals, therapies and interventions which, when administered to a patient with an immunosuppressive agent, can be effective in treating the immune disorders of the present invention. In addition, as described in detail, below, such animal models can be used to determine the LD₅₀ and the ED₅₀ in animal subjects, and such data can be used to determine the in vivo efficacy of potential immune disorder treatments.

### 5.5.1.1 Animal-Based Systems

Animal-based model systems of immune disorders can include both non-recombinant animals as well as recombinantly engineered transgenic animals.

Animal models for TH cell subpopulation-related disorders can include, for example, genetic models. For example, such animal models can include Leishmania resistance models, experimental allergic encephalomyelitis models and (BALB/c Cr x DBA/2Cr) F1 mice. These latter mice develop a fatal disseminated disease by systemic infection with virulent Candida albicans associated with strong TH2-like responses. Well known mouse models for asthma can be utilized to study the amelioration of symptoms caused in immune disorders, such as allergy and asthma, that are associated with a strong TH2 or TH2-like response. (See, for example, Lukacs et al., 1994, Am. J. Resp. Cell Mol. Biol. 10:526-532; Gavett et al., 1994, Am. J. Resp. Cell Mol. Biol. 10:587-593.) Further, the animal model, murine acquired immunodeficiency syndrome (MAIDS; Kanagawa et al., 1993, Science 262:240; Makino et al., 1990, J. 1 mm. 144:4347) can be used for such studies.

Alternatively, such well known animal models as SCIDhu mice (see for example, H. Kaneshima et al., 1994, Curr. Opin. Imm. 6:327-333) which represents an in vivo model of the human hematolymphoid system, can be utilized. Further, the RAG-2-deficient blastocyst complementation technique (J. Chen et al., 1993, Proc. Natl. Acad. Sci. USA 90:4528-4532; Y. Shinkai et al., 1992, Cell 68:855-867) can be utilized to produce mice containing, for example, humanized lymphocytes and/or which express target gene sequences. Still further, targeting techniques directed specifically to T cells, for example, the technique of Gu et al. (1994, Science 265:103-106) can be utilized to produce animals containing transgenes in only T cell populations.

Further, animal models such as the adoptive transfer model described, e.g., in L. Cohn et al., 1997, J. Exp. Med. 186:1737-1747) below, can be used. In such an animal system, aeroallergen provocation of TH1 or TH2 recipient mice results in TH effector cell migration to the airways and is associated with an intense neutrophilic (TH1) and eosinophilic (TH2) lung mucosal inflammatory response. The animal model represents an accepted model for asthma.

In a preferred embodiment, the animal model for testing the efficacy of the HVEM-LIGHT inhibitor, e.g., an inhibitor identified by the in vitro-based methods described above, is an allograft rejection, most preferably a cardiac allograft rejection, murine model.

Animals models for other immune disorders are known in the art. TH1-related disorder symptoms can include, for example, those associated with chronic inflammatory diseases and disorders, such as Crohn's disease, reactive arthritis (including but not limited to Lyme disease), insulin-dependent diabetes, organ-specific autoimmunity, (including but not limited to multiple sclerosis, Hashimoto's thyroiditis and Grave's disease), contact dermatitis, psoriasis, graft rejection, graft versus host disease and sarcoidosis to name a few. TH2-related disorder symptoms can include, but are not limited to, those associated with atopic conditions such as asthma and allergy such as allergic rhinitis, gastrointestinal allergies, (including but not limited to food allergies); eosinophilia; conjunctivitis; glomerular nephritis; certain pathogen susceptibilities such as helminthic (e.g., leishmaniasis); and certain viral infections, including HIV, and bacterial infections (for example, tuberculosis and lepromatous leprosy).

Additionally, cellular phenotypes characteristic of the immune disorders of the invention can be assayed for efficacy of the HVEM-LIGHT inhibitor and immunosuppressive agent combinatorial therapy. Such cellular phenotypes can include, for example, cytokine expression profiles, which, are assayed for reversion from the disease state to the normal states upon administration of the HVEM-LIGHT inhibitor and immunosuppressive agent in accordance with the methods of the invention, for example as described in Section 6.2 below.

### 5.5.1.2 Cell-Based Assays

Cell populations which endogenously or recombinantly express HVEM and LIGHT proteins can be utilized to identify or validate potential HVEM-LIGHT inhibitors. Cellular phenotypes which can indicate inhibition of the LIGHT-HVEM interaction can include, for example, an inhibition of cytokine or cell surface marker expression associated with LIGHT-HVEM signaling.

In order to recombinantly express a LIGHT or HVEM protein, the LIGHT or HVEM open reading frame, respectively, can be ligated to a regulatory sequence which is capable of driving gene expression in the cell type of interest. Such regulatory regions will be well known to those of skill in the art, and can be utilized in the absence of undue experimentation. Transfection of the LIGHT or HVEM expression construct can be accomplished by utilizing standard techniques. See, for example, Ausubel, 1989, supra. Transfected cells should be evaluated for the presence of the recombinant target gene sequences, for expression and accumulation of target gene mRNA, and for the presence of recombinant target gene protein production.

5.6 Pharmaceutical Preparations and Methods of Administration

The immunosuppressive agents and HVEM-LIGHT inhibitors that are useful in the present methods and compositions, such as those described herein, can be administered to a patient in amounts effective to treat or prevent allograft rejection. As used herein, “amounts effective to treat or prevent allograft rejection” refers to the combined amount of HVEM-LIGHT inhibitor and immunosuppressive agent.

### 5.6.1 Effective Dose

The present invention provides the benefit of administration of reduced amounts of the HVEM-LIGHT inhibitor and the immunosuppressive agent relative what would be required for each of the compounds alone to achieve a therapeutic or prophylactic effect, thereby lowering the toxicity associated with administration of such compounds. Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects can be used, care should be taken to design a delivery system that targets such compounds to the site of affected tissue in order to minimize potential damage to unaffected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured by any technique known in the art, for example, by high performance liquid chromatography.

For compounds whose therapeutic dose in single therapy regimens has been determined, it is preferable to use dosages at the lower end of or below the determined therapeutic dose, although higher doses can be used, for example where single therapy is not successful. For example, the therapeutic dose of cyclosporine A for treating graft rejection in humans is in the range of 2 to 6 mg/kg/day. Thus, in the present methods and compositions, cyclosporine A can be used in amounts of, for example, 0.5-10 mg/kg/day, more preferably 1-5 mg/kg/day, and most preferably 2-3 mg/kg/day.

### 5.6.2 Formulations and use

The invention relates to pharmaceutical compositions and methods of use thereof for preventing or treating allograft rejection. Such pharmaceutical compositions can be formulated in a conventional manner using one or more physiologically acceptable carriers or excipients. In one embodiment of the present invention, the HVEM-LIGHT inhibitor and the immunosuppressive agent are formulated into one pharmaceutical composition, most preferably in amounts that are effective to treat or prevent an immune disorder such as allograft rejection.

Thus, the compounds and their physiologically acceptable salts and solvents can be formulated for systemic administration or local administration at the site of immune dysfunction. Further, the compounds can be formulated for administration by inhalation or insufflation (either through the mouth or the nose) or oral, buccal, parenteral or rectal administration.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds can be formulated for parenteral administration (i.e., intravenous or intramuscular) by injection, via, for example, bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

5.7 Kits

The present invention provides kits for practicing the methods of the present invention. A kit of the invention comprises in one or more containers an HVEM-LIGHT inhibitor, such as those described in Section 5.2, supra, or an immunosuppressive agent, such as those described in Section 5.3, supra.

The kit of the invention may optionally comprise additional components useful for performing the methods of the invention. By way of example, the kit may comprise pharmaceutical carriers useful for formulating the HVEM-LIGHT inhibitor and/or the immunosuppressive agent. Where the HVEM-LIGHT inhibitor or immunosuppressive agent is administered in the form of cell therapy or gene therapy, suitable cells or gene therapy vectors may also be included. In addition, the kits of the invention may further provide an instructional material which describes performance of the methods of the inveniton, or a notice in the form prescribed by a governmental agency regulating the manufacture, use or sale of pharmaceuticals or biological products, which notice reflects approval by the agency of manufacture, use or sale for human administration.

5.8 Murine HVEM Gene Sequences

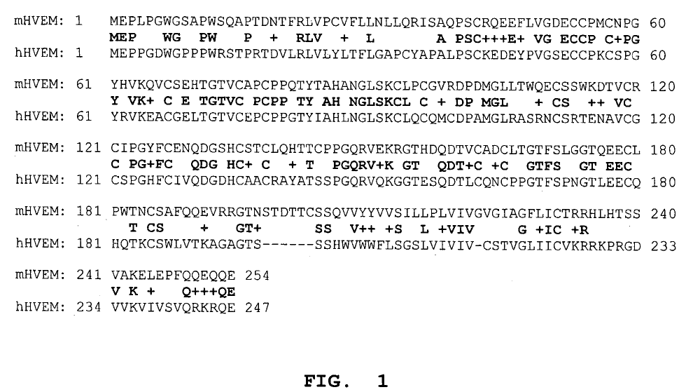

The present invention discloses novel murine HVEM nucleic acids and polypeptides. Prior to the present disclosure, the full length murine cDNA and its encoded protein had not been characterized. As shown in FIG. 1, the murine and human sequences share 46% sequence identify and 57% sequence similarity, with the greatest stretch of identity being seven consecutive amino acids at positions 88-94.

### 5.8.1 mHVEM Nucleic Acids

The present invention provides murine HVEM nucleic acids. The murine HVEM cDNA is represented by SEQ ID NO:3.

In one embodiment, the present invention provides an isolated nucleic acid molecule having a nucleotide sequence which is at least 65%, 70%, 75%, 80%, 85%, 90%, 95% or 98% identical to the nucleotide sequence of SEQ ID NO:3 or a complement thereof as determined using the BLAST algorithm. In another embodiment, the present invention provides an isolated nucleic acid molecule comprising at least 15, 20, 30, 40 or 50 nucleotide residues and having a nucleotide sequence identical to at least 15, 20, 30, 40 or 50 consecutive nucleotide residues of residues 1-424 of SEQ ID NO:3 or a complement thereof. In another embodiment, the present invention provides an isolated nucleic acid molecule which encodes a polypeptide comprising the amino acid sequence of SEQ ID NO:4. In yet another embodiment, the present invention provides an isolated nucleic acid molecule which encodes a fragment of a polypeptide comprising the amino acid sequence of any of SEQ ID NO:4, wherein the fragment comprises at least 10, 12, 15, 20, 25, 30, 40 or 50 consecutive amino acid residues of residues 1-80 of SEQ ID NO:4. In yet another embodiment, the present invention provides an isolated nucleic acid molecule which encodes a naturally occurring allelic variant of a polypeptide comprising the amino acid sequence of SEQ ID NO:4, wherein the nucleic acid molecule hybridizes under stringent conditions with a nucleic acid molecule consisting of the nucleotide sequence of any of residues 1-424 of SEQ ID NO:3 or a complement thereof. Preferably, the foregoing nucleic acids encode a polypeptide that has one or more characteristics of mHVEM. Such characteristics include the ability to bind to gD, LIGHT, or Ltα; or the ability to immunospecifically bind to an antibody that immunospecifically binds to mHVEM.

Further, the present invention provides an isolated nucleic acid having the nucleotide sequence of SEQ ID NO:3 or a complement thereof. The present invention also provides an isolated nucleic acid molecule which encodes a polypeptide having the amino acid sequence of any of SEQ ID NO:4 or a complement thereof.

Nucleic acids encoding any of the mHVEM polypeptides described in Section 5.8.2 below are also provided.

In a preferred embodiment, the foregoing nucleic acids further comprise vector nucleic acid sequences and/or nucleic acid sequences encoding a heterologous polypeptide.

The present invention further provides mammalian and non-mammalian host cells containing any of the foregoing mHVEM nucleic acid molecules. The host cell can be a mammalian or non-mammalian host cell.

### 5.8.2 mHVEM Polypeptides

The present invention provides murine HVEM polypeptides. The murine HVEM open reading frams is represented by SEQ ID NO:4.

The present invention provides an isolated polypeptide comprising a fragment of a polypeptide comprising the amino acid sequence of SEQ ID NO:4, wherein the fragment comprises at least 10, 12, 15, 20, 25, 30, 40 or 50 consecutive amino acid residues of residues 1-80 of SEQ ID NO:4. The present invention further provides an isolated polypeptide comprising naturally occurring allelic variant of a polypeptide comprising the amino acid sequence of any of SEQ ID NO:4, wherein the polypeptide is encoded by a nucleic acid molecule which hybridizes with a nucleic acid molecule consisting of the nucleotide sequence of residues 1-424 of SEQ ID NO:3 or a complement thereof under stringent conditions. Yet further, the present invention provides a polypeptide which is encoded by a nucleic acid molecule comprising a nucleotide sequence which is at least 65%, 70%, 75%, 80%, 85%, 90%, 95% or 98% identical to a nucleic acid consisting of residues 1-424 of SEQ ID NO:3 or a complement thereof as determined using the NBLAST algorithm. Polypeptides encoded by any of the mHVEM nucleic acids described in Section 5.8.1, supra, are also provided.

In a specific embodiment, the present invention provides a polypeptide having the amino acid sequence of SEQ ID NO:4.

The present invention yet further provides a polypeptide, preferably an isolated polypeptide, comprising any of the foregoing mHVEM polypeptides fused at its amino or carboxy terminal to heterologous amino acid residues. Suitable fusion partners for mHVEM polypeptides are described in Section 5.2.1.1., supra.

The present invention further provides antibodies which selectively bind to murine HVEM polypeptides, but not to human HVEM polypeptides. Such antibodies can be made by the methods described in Section 5.2.2. above.

The present invention further provides methods of making mHVEM polypeptides, comprising culturing a host cell comprising a recombinant nucleic acid that encodes a mHVEM polypeptide operably linked to a promoter under conditions such that the mHVEM polypeptide is expressed.

The following experimental examples are offered by way of illustration and not by way of limitation.

### 6. EXAMPLE

### Modulation of LIGHT-HVEM Costimulation Prolongs Cardiac Allograft Survival

LIGHT, a TNF superfamily member expressed by activated T cells, binds to herpesvirus entry mediator (HVEM) which is constitutively expressed by T cells. LIGHT costimulates T cell activation in a CD28-independent manner, and given interest in regulating the effector functions of T cells in vivo, the role of LIGHT-HVEM costimulation was examined in a murine cardiac allograft rejection model. Normal hearts lacked LIGHT or HVEM mRNA expression, but allografts showed strong expression of both genes from day 3 post-transplant, and in-situ hybridization and immunohistology localized LIGHT and HVEM to infiltrating leukocytes. To test the importance of LIGHT expression on allograft survival, we generated LIGHT−/− mice by homologous recombination. The survival of fully MHC-mismatched cardiac allografts was longer in LIGHT−/− vs. LIGHT+/+ mice (p<0.05), and LIGHT−/− allograft recipients treated with cyclosporin A (CsA) maintained their allografts longer than untreated LIGHT−/− mice (p<0.001) or CsA-treated wild-type (LIGHT+/+) controls (p<0.001). Molecular analyzes showed that the beneficial effects of targeting of LIGHT were accompanied by decreased intragraft expression of IFN-γ, plus IFN-γ-induced chemokine, IP-10, and its receptor, CXCR3. Further evidence in support of a role of this pathway in alloresponses was shown by the beneficial effects on allograft survival in LIGHT+/+ allograft recipients treated with HVEM-Ig plus CsA. The data presented herein indicates that LIGHT/HVEM costimulation is a significant component of the host response leading to cardiac allograft rejection.

6.1 Materials and Methods

Generation of LIGHT−/− Mice. A targeting vector was constructed using a 10.5-kb genomic fragment containing exons 1-4 of the LIGHT gene. A 0.8 kb sequence around exon 1, including ATG, was deleted and replaced by pMC1 neo, the targeting vector was linearized and electroporated into ES cells, and LIGHT+/− ES cell clones were selected in media containing G418 and Gancyclovir, as described (Hancock et al., 2000, J Exp Med 192:1515-20). The correctly targeted event was screened by Southern blotting, and chimeric mice were derived by blastocyst injection. Offspring of LIGHT+/− mice were crossed to produce LIGHT−/− mice. Mice used in this study were on a B6/129 background.

Transplantation. Male 6-week-old BALB/c (H-2d), 129Sv/J (H-2^{b}), C57BL/6 (H-2^{b}) and B6/129 (H-2^{b}) mice were purchased from The Jackson Laboratory and maintained in our specific pathogen-free facility. Heterotopic abdominal cardiac allografting was done with the use of BALB/c donors and B6/129 recipients, as well as in some experiments, pure 129 or B6 recipients, using 6 allografts per experimental group (Hancock et al., 1996, Proc Natl Acad Sci USA 93:13967-72); data reported are from BALB->B6/129 unless noted otherwise. Allograft function was monitored twice daily by palpation, and rejection confirmed by laparotomy and histologic examination. Upon harvest at rejection or at the time indicated, mid-ventricular samples were fixed in formalin for light microscopy or snap-frozen in liquid nitrogen and stored at −80° C. for subsequent immunohistology and RNA studies.

Post-transplant Therapies. LIGHT−/− or LIGHT+/+ allograft recipients were treated with cyclosporin A (CsA) (Sigma), dissolved in olive oil and administered daily (10 mg/kg i.p.) for 14 d post-transplant. Additional LIGHT+/+ recipients were treated with a mHVEM-human IgG1 (HVEM-Ig) fusion protein, which was generated using the, extracellular region of HVEM (Mauri et al., 1998, Immunity 8:21-30); recipients received 100 μg of HVEM-Ig daily, from the time of transplantation until day 14.

RNA Isolation and RT-PCR Analysis of HVEM and LIGHT Expression. Total RNA was prepared from each recipient's heart and cardiac allograft using guanidine-thiocyanate, and RNA integrity was confirmed by electrophoresis (Chomczynski and Sacchi, 1987, Anal. Biochem. 162:156-9). RNA was reverse transcribed at 45° C. for 60 min, 95° C. for 3 min, and placed on ice. For LIGHT detection, primers (mLIGHT1F, 5-ATGGAGAGTGTGGTACAGCCTTC-3 (SEQ ID NO:20); mLIGHT1R, 5-GACCATGAAAGCTCCGAAATAGG-3 (SEQ ID NO:21)) were used. For HVEM, primers (mHVEM1F, 5-ATGGAACCTCTCCCAGGATGGG-3 (SEQ ID NO:22); mHVEM1R, 5-TCAGTTGGAGGCTGTCTCCTCC-3 (SEQ ID NO:23)) were used. Each 3-step thermal cycle for routine PCR analysis included 30 s at 95° C., 30 s at 60° C., and 60 s at 72° C.; additional PCR reactions were performed for 35 cycles. PCR products were visualized by ethidium bromide staining of agarose gels and identified by size markers. A negative control containing all reagents except cDNA was included in each PCR analysis.

In-situ Hybridization (ISH). LIGHT riboprobes were synthesized from T3 (sense probe) and T7 (anti-sense probe) promoters, labeled with biotin-UTP (Roche) and used to localize LIGHT mRNA expression within cardiac tissue sections by ISH, as described (Gao et al., 2000, J Clin Invest 105:35-44).

RNase Protection Assays. RNA was evaluated by the Riboquant system (PharMingen), using mouse template sets mCK-1 and mCK-3b for cytokines; mCK5 for chemokines; and mCR5 and mCR6 for CC and CXC chemokine receptors, respectively. A riboprobe for mouse CXCR3 was prepared in house (Topham et al., 1999, J Clin Invest 104:1549-57). Methods for in vitro transcription, riboprobe purification and use, plus densitometric analysis and normalization of data to L32 and GAPDH gene expression, were as described (Gao et al., 2000, J Clin Invest 105:35-44).

Immunopathology. Hearts were fixed in formalin, paraffin-embedded, and stained with hematoxylin and eosin. Cryostat sections fixed in paraformaldehyde-lysine-periodate were stained by immunoperoxidase using mAbs to mouse leukocytes (Pharmingen), anti-LIGHT antibody (Santa Cruz Biotechnology) or isotype-matched controls (Hancock et al, 1996, Proc Natl Acad Sci USA 93:13967-72).

6.2 Results and Discussion

Given the importance of LIGHT expression in the development of T cell activation in vivo, including in host T cell-dependent anti-tumor responses (Tamada et al., 2000, Nat. Med. 6:283-9; Zhai et al, 1998, J. Clin. Invest. 102:1142-51), we undertook a serial study of LIGHT and HVEM expression during the development of cardiac allograft rejection across a full MHC disparity. Total RNA was prepared from each recipient's heterotopic transplant and own native heart after collection on days 3 and 7 post-transplant, and HVEM and LIGHT mRNA expression were analyzed by RT-PCR. Negligible LIGHT or HVEM mRNA was detected in native hearts, but Northern analysis of LIGHT and HVEM expression in serial cardiac transplants rejected by day 7 indicated that expression of both LIGHT and HVEM were markedly upregulated at days 3 and 7 post-transplant in comparison with LIGHT and HVEM expression in the recipient's native heart. In situ hybridization analysis showed LIGHT mRNA expression in the graft LIGHT at day 7 post-transplant to be restricted to focal mononuclear cells with the morphology of leukocytes. HVEM mRNA was similarly localized to focal mononuclear cells; in addition, focal endothelial expression of HVEM was observed. Immunoperoxidase studies showed staining of infiltrating mononuclear leukocytes by anti-LIGHT IgG, including large cells with the morphologic features of inflammatory macrophages at day 7 post-transplant, including in cell clumps containing larger, branching DC-like cells.

To determine the role of LIGHT expression in allograft rejection, exon 1 of the LIGHT gene, which contains the ATG initiation codon, was disrupted by homologous recombination. A LIGHT gene-targeting construct was generated to replace exon 1 of LIGHT containing the initiating methionine (ATG) with the neomycin resistance gene (NEO). Southern analysis of LIGHT wild-type (+/+), heterozygous knockout (+/−) and homozygous knockout (−/−) mouse genomic DNA confirmed the replacement of exon 1 by the targeting construct. Northern analysis of LIGHT wild-type (+/+) and homozygous knockout (−/−) mouse liver RNA using a probe containing the entire coding region of LIGHT showed no detectable LIGHT mRNA expression in the LIGHT−/− liver tissue.

Mice heterologous and homozygous for the LIGHT mutation were normal in appearance, growth and fertility; had normal numbers of T and B cells, monocytes and granulocytes; and had normal lymphoid architecture by light microscopy.

Homozygous LIGHT−/− mice were used as recipients of fully MHC-disparate cardiac allografts. Whereas LIGHT+/+ mice rejected BALB/c cardiac allografts within 1 wk, LIGHT−/− mice maintained their grafts for an extra 3-4 days, which was about as effective as a sub-therapeutic dose of CsA (10 mg/kg/d) in mice (both p<0.05 vs. untreated LIGHT+/+ recipients). However, use of the same regimen of CsA in LIGHT−/− mice led to significantly prolonged engraftment (˜30 d, p<0.001), indicating a synergistic effect of CsA and LIGHT targeting on allograft survival.

Given concerns that gene-targeted mice might not always accurately reflect the full immune repertoire, the effect of inhibiting the HVEM-LIGHT pathway was also studied in wild-type allograft recipients. Accordingly, an HVEM-Ig fusion protein was constructed for therapeutic blockade of the effects of endogenous LIGHT on host HVEM+ T cells. Consistent with the effects of LIGHT targeting by homologous recombination in this strong MHC disparity, the studies in the wild-type allograft recipients demonstrated that HVEM-Ig alone or a combination of IgG and a subtherapeutic dose of CsA had negligible effects on allograft survival in LIGHT+/+ recipients (p>0.05). In contrast, the combination of HVEM-Ig plus low-dose CsA was markedly synergistic, significantly prolonging cardiac allograft survival (P<0.001).

The levels of expression of multiple cytokines, chemokines and their receptors by host leukocytes vary during transplant rejection. RNase protection assays were used to examine the likely mechanisms by which targeting of LIGHT, especially in the context of concomitant CsA usage, induced prolonged allograft survival. The expression of intragraft Th1 and Th2-associated cytokines in control normal heart and allografts harvested at day 7 post-transplant was compared. Relative to expression in normal heart tissue, expression of IL-10, IFN γ, and IL-2 was upregulated in allograft recipients, including in LIGHT-deficient allograft recipients and allograft recipients treated with CsA at a dose of 10 mg/kg/day. Treatment of LIGHT−/− mice with CsA suppressed the intragraft upregulation of multiple cytokine mRNAs, including IFN-γ, IL-2 and IL-10, as well as LT-β and TNF-α In contrast, expression of TGF-2 was upregulated upon treatment of LIGHT−/− allograft recipients with subtherapeutic doses of CsA.

Consistent with these effects, expression of several IFN-γ-induced chemokines, including RANTES, MIP-1α, MIP-1β and IP-10, which are upregulated in cardiac tissue of allograft recipients receiving no therapy or single therapy relative to normal cardiac tissue, were markedly down-regulated in LIGHT−/− mice treated with CsA. Moreover, along with the decreased chemokine expression, LIGHT−/− mice treated with CsA had modest reductions in CC-chemokine receptor expression and markedly decreased expression of the chemokine receptor for IP-10, CXCR3, which is expressed by Th1 and Tcl lymphocytes (Hancock et al., 2000, J. Exp. Med. 192:1515-20). Prior studies in this model showed that donor-derived IP-10 production (Hancock et al., 2001, J. Exp. Med. 193:975-80) and concomitant infiltration by CXCR3+leukocytes (Hancock et al., 2000, J. Exp. Med. 192:1515-20) play central roles in the development of allograft rejection, such that targeting of either IP-10 production or CXCR3 expression markedly prolongs allograft survival. The IP-10/CXCR3 pathway is active during development of human cardiac allograft rejection (Melter et al., 2001, Circulation 104:2558-64). Thus, the combinatorial therapy methods described in the present study lead to a reduction of various molecular parameters that are indicative of allograft rejection.

The studies presented herein show marked synergism by combining immunosuppressive agents and blockade of HVEM-LIGHT costimulation. This is in contrast with other combination therapies, such as the use of CD154 mAb plus CsA (Smiley et al., 2000, Transplantation 70:415-9). As discussed above, the combination therapy methods of the present invention prevent modulate intragraft cytokine and chemokine production and decrease the infiltration of host immunocompetent cells, thereby inhibiting allograft rejection and prolonging survival of allograft recipients.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications are cited herein, the disclosures of which are incorporated by reference in their entireties.

What is claimed is:

1. A method of treating or preventing an immune disorder in a patient, comprising administering to the patient (i) an HVEM-LIGHT inhibitor; and (ii) an immunosuppressive agent, in amounts effective for treating or preventing the immune disorder.

2. The method of claim 1, wherein the immune disorder is allograft rejection, delayed-type hypersensitivity, graft versus host disease, a drug allergy, atrophic gastrititis, thyroiditis, allergic encephalomyelitis, autoimmune hemolytic anemia, sympathetic ophthalmia, systemic lupus erythematosus, rheumatoid arthritis, multiple sclerosis, asthma, inflammatory bowel disease, or myasthenia gravis.

3. The method of claim 2, wherein the immune disorder is allograft rejection.

4. The method of claim 3, wherein the allograft is an organ transplant.

5. The method of claim 4, wherein the organ is a heart, kidney, liver, lung or pancreas.

6. The method of claim 3, wherein the allograft is a tissue transplant.

7. The method of claim 6, wherein the tissue is heart valve, endothelial, cornea, eye lens or bone marrow tissue.

8. The method of claim 3, wherein the allograft is a skin graft.

9. The method of claim 1 or 3, wherein the immunosuppressive agent is cyclosporine.

10. The method of claim 1 or 3, wherein the immunosuppressive agent is FK506.

11. The method of claim 1 or 3, wherein the immunosuppressive agent is a steroid.

12. The method of claim 11, wherein the steroid is a corticosteroid.

13. The method of claim 1 or 3, wherein the immunosuppressive agent is rapamycin.

14. The method of claim 1 or 3, wherein the immunosuppressive agent is an antibody.

15. The method of claim 1 or 3, wherein the immunosuppressive agent is an antiproliferative agent.

16. The method of claim 15, wherein the antiproliferative agent is azathioprine or mycophenolate moefitil.

17. The method of claim 1 or 3, wherein the immunosuppressive agent is prednisone.

18. The method of claim 1 or 3, wherein the immunosuppressive agent is a purine analog.

19. The method of claim 18, wherein the purine analog is methotrexate or mercaptopurine.

20. The method of claim 1 or 3, wherein the HVEM-LIGHT inhibitor and the immunosuppressive agent are administered concurrently.

21. The method of claim 20, wherein the HVEM-LIGHT inhibitor and the immunosuppressive agent are both administered daily.

22. The method of claim 1 or 3, wherein the HVEM-LIGHT inhibitor and the immunosuppressive agent are administered successively.

23. The method of claim 22, wherein the HVEM-LIGHT inhibitor is administered prior to administration of the immunosuppressive agent.

24. The method of claim 22, wherein the HVEM-LIGHT inhibitor is administered following administration of the immunosuppressive agent.

25. The method of claim 22, wherein the HVEM-LIGHT inhibitor and the immunosuppressive agent are administered at a 1-day interval.

26. The method of claim 22, wherein the HVEM-LIGHT inhibitor and the immunosuppressive agent are administered at a 2-day interval.

27. The method of claim 22, wherein the HVEM-LIGHT inhibitor and the immunosuppressive agent are administered at a 3-day interval.

28. The method of claim 1 or 3, wherein the HVEM-LIGHT inhibitor is a soluble HVEM polypeptide.

29. The method of claim 28, wherein the soluble HVEM polypeptide is a fusion protein further comprising an Ig domain.

30. The method of claim 1 or 3, wherein the HVEM-LIGHT inhibitor is a soluble LIGHT polypeptide.

31. The method of claim 30, wherein the soluble LIGHT polypeptide is a fusion protein further comprising an Ig domain.

32. The method of claim 1 or 3, wherein the HVEM-LIGHT inhibitor is not a herpesvirus gD protein, a lymphotoxin-α protein, a lymphotoxin-β receptor protein, or a TR6 receptor protein.

33. The method of claim 1 or 3, wherein the HVEM-LIGHT inhibitor is an anti-HVEM antibody.

34. The method of claim 1 or 3, wherein the HVEM-LIGHT inhibitor is an anti-LIGHT antibody.

35. The method of claim 1 or 3, wherein the HVEM-LIGHT inhibitor is administered in the form of gene therapy.

36. The method of claim 1 or 3, wherein the amounts of the HVEM-LIGHT inhibitor and immunosuppressive agent are synergistic.

37. The method of claim 36, wherein the amount of the HVEM-LIGHT inhibitor or immunosuppressive agent, if administered alone, is not effective for treating allograft rejection.

38. The method of claim 36, wherein the amount of each of the HVEM-LIGHT inhibitor and immunosuppressive agent, if administered alone, is not effective for treating allograft rejection.

39. The method of claim 1 or 3, wherein the HVEM-LIGHT inhibitor is conjugated to the immunosuppressive agent.

40. A pharmaceutical composition comprising:
a. an HVEM-LIGHT inhibitor;
b. an immunosuppressive agent; and
c. a pharmaceutically acceptable carrier;
wherein the HVEM-LIGHT inhibitor and the immunosuppressive agent are present in amounts effective to prevent or treat allograft rejection.

41. A kit comprising in one or more containers:
a. HVEM-LIGHT inhibitor;
b. an immunosuppressive agent; and
c. instructions for the use of the HVEM-LIGHT inhibitor and the immunosuppressive agent in the prevention or treatment of allograft rejection.

42. An isolated nucleic acid molecule selected from the group consisting of:
a. a nucleic acid molecule having a nucleotide sequence which is at least 90% identical to the nucleotide sequence of SEQ ID NO:3 or a complement thereof as determined using the BLAST algorithm;
b. a nucleic acid molecule comprising at least 15 nucleotide residues and having a nucleotide sequence identical to at least 15 consecutive nucleotide residues of residues 1-424 of SEQ ID NO:3 or a complement thereof;
c. a nucleic acid molecule which encodes a polypeptide comprising the amino acid sequence of SEQ ID NO:4;
d. a nucleic acid molecule which encodes a fragment of a polypeptide comprising the amino acid sequence of any of SEQ ID NO:4, wherein the fragment comprises at least 12 consecutive amino acid residues of residues 1-80 of SEQ ID NO:4; and
e. a nucleic acid molecule which encodes a naturally occurring allelic variant of a polypeptide comprising the amino acid sequence of SEQ ID NO:4, wherein the nucleic acid molecule hybridizes under stringent conditions with a nucleic acid molecule consisting of the nucleotide sequence of any of residues 1-424 of SEQ ID NO:3 or a complement thereof.

43. The isolated nucleic acid molecule of claim 42, which is selected from the group consisting of:
a. a nucleic acid having the nucleotide sequence of SEQ ID NO:3 or a complement thereof; and
b. a nucleic acid molecule which encodes a polypeptide having the amino acid sequence of any of SEQ ID NO:4 or a complement thereof.

44. The isolated nucleic acid molecule of claim 44, wherein the fragment comprises at least 15 consecutive amino acid residues of residues 1-80 of SEQ ID NO:4.

45. The isolated nucleic acid molecule of claim 44, wherein the fragment comprises at least 20 consecutive amino acid residues of residues 1-80 of SEQ ID NO:4.

46. The isolated nucleic acid molecule of claim 44, wherein the fragment comprises at least 30 consecutive amino acid residues of residues 1-80 of SEQ ID NO:4.

47. The isolated nucleic acid molecule of claim 44, wherein the fragment comprises at least 40 consecutive amino acid residues of residues 1-80 of SEQ ID NO:4.

48. The isolated nucleic acid molecule of claim 44, wherein the fragment comprises at least 50 consecutive amino acid residues of residues 1-80 of SEQ ID NO:4.

49. The isolated nucleic acid molecule of claim 42, 43, or 44, further comprising vector nucleic acid sequences.

50. The isolated nucleic acid molecule of claim 42, 43, or 44, further comprising nucleic acid sequences encoding a heterologous polypeptide.

51. A host cell which contains the nucleic acid molecule of claim 42, 43, or 44.

52. The host cell of claim 51 which is a mammalian host cell.

53. The host cell of claim 51 which is a non-mammalian host cell.

54. An isolated polypeptide selected from the group consisting of:
a. a fragment of a polypeptide comprising the amino acid sequence of SEQ ID NO:4; wherein the fragment comprises at least 12 consecutive amino acid residues of residues 1-80 of SEQ ID NO:4;
b. a naturally occurring allelic variant of a polypeptide comprising the amino acid sequence of any of SEQ ID NO:4, wherein the polypeptide is encoded by a nucleic acid molecule which hybridizes with a nucleic acid molecule consisting of the nucleotide sequence of residues 1-424 of SEQ ID NO:3 or a complement thereof under stringent conditions; and
c. a polypeptide which is encoded by a nucleic acid molecule comprising a nucleotide sequence which is at least 90% identical to a nucleic acid consisting of residues 1-424 of SEQ ID NO:3 or a complement thereof as determined using the BLAST algorithm.

55. The isolated polypeptide of claim 54 having the amino acid sequence of SEQ ID NO:4.

56. The isolated polypeptide of claim 54, wherein the fragment comprises at least 15 consecutive amino acid residues of residues 1-80 of SEQ ID NO:4.

57. The isolated polypeptide of claim 56, wherein the fragment comprises at least 20 consecutive amino acid residues of residues 1-80 of SEQ ID NO:4.

58. The isolated polypeptide of claim 56, wherein the fragment comprises at least 30 consecutive amino acid residues of residues 1-80 of SEQ ID NO:4.

59. The isolated polypeptide of claim 56, wherein the fragment comprises at least 40 consecutive amino acid residues of residues 1-80 of SEQ ID NO:4.

60. The isolated polypeptide of claim 56, wherein the fragment comprises at least 50 consecutive amino acid residues of residues 1-80 of SEQ ID NO:4.

61. The isolated polypeptide of claim 54, wherein the amino acid sequence of the polypeptide further comprises heterologous amino acid residues.

62. An antibody which selectively binds with the polypeptide of claim 54, which antibody does not bind to a polypeptide comprising SEQ ID NO:2.

63. A method for producing a polypeptide selected from the group consisting of:
a. a fragment of a polypeptide comprising the amino acid sequence of SEQ ID NO:4; wherein the fragment comprises at least 12 consecutive amino acid residues of residues 1-80 of SEQ ID NO:4;
b. a naturally occurring allelic variant of a polypeptide comprising the amino acid sequence of any of SEQ ID NO:4, wherein the polypeptide is encoded by a nucleic acid molecule which hybridizes with a nucleic acid molecule consisting of the nucleotide sequence of residues 1-424 of SEQ ID NO:3 or a complement thereof under stringent conditions; and
c. a polypeptide which is encoded by a nucleic acid molecule comprising a nucleotide sequence which is at least 90% identical to a nucleic acid consisting of residues 1-424 of SEQ ID NO:3 or a complement thereof as determine using the BLAST algorithm.
said method comprising culturing the host cell of claim 51 under conditions in which the nucleic acid molecule is expressed.


SEQUENCE LISTING (23 sequences)

SEQ ID NO: 1 - DNA, 1724 bp, Homo sapiens
  CDS (294)..(1145)
ccttcataccggcccttcccctcggctttgcctggacagctcctgcctcccgcagggcccacctgtgtcccccagcgccgctccacccagcaggcctgagcccctctctgctgccagacaccccctgctgcccactctcctgctgctcgggttctgaggcacagcttgtcacaccgaggcggattctctttctctttctcttctggcccacagccgcagcaatggcgctgagttcctctgctggagttcatcctgctagctgggttcccgagctgccggtctgagcctgaggcatggagcctcctggagactgggggcctcctccctggagatccacccccagaaccgacgtcttgaggctggtgctgtatctcaccttcctgggagccccctgctacgccccagctctgccgtcctgcaaggaggacgagtacccagtgggctccgagtgctgccccaagtgcagtccaggttatcgtgtgaaggaggcctgcggggagctgacgggcacagtgtgtgaaccctgccctccaggcacctacattgcccacctcaatggcctaagcaagtgtctgcagtgccaaatgtgtgacccagccatgggcctgcgcgcgagccggaactgctccaggacagagaacgccgtgtgtggctgcagcccaggccacttctgcatcgtccaggacggggaccactgcgccgcgtgccgcgcttacgccacctccagcccgggccagagggtgcagaagggaggcaccgagagtcaggacaccctgtgtcagaactgccccccggggaccttctctcccaatgggaccctggaggaatgtcagcaccagaccaagtgcagctggctggtgacgaaggccggagctgggaccagcagctcccactgggtatggtggtttctctcagggagcctcgtcatcgtcattgtttgctccacagttggcctaatcatatgtgtgaaaagaagaaagccaaggggtgatgtagtcaaggtgatcgtctccgtccagcggaaaagacaggaggcagaaggtgaggccacagtcattgaggccctgcaggcccctccggacgtcaccacggtggccgtggaggagacaataccctcattcacggggaggagcccaaaccactgacccacagactctgcaccccgacgccagagatacctggagcgacggctgctgaaagaggctgtccacctggcgaaaccaccggagcccggaggcttgggggctccgccctgggctggcttccgtctcctccagtggagggagaggtggggcccctgctggggtagagctggggacgccacgtgccattcccatgggccagtgagggcctggggcctctgttctgctgtggcctgagctccccagagtcctgaggaggagcgccagttgcccctcgctcacagaccacacacccagccctcctgggccagcccagagggcccttcagaccccagctgtctgcgcgtctgactcttgtggcctcagcaggacaggccccgggcactgcctcacagccaaggctggactgggttggctgcagtgtggtgtttagtggataccacatcggaagtgattttctaaattggatttgaattccggtcctgtcttctatttgtcatgaaacagtgtatttggggagatgctgtgggaggatgtaaatatcttgtttctcctcaaaaaaaaaaaaaaaaaaaaaaaaaa

SEQ ID NO: 2 - protein, 283 aa, Homo sapiens [sequence omitted]

SEQ ID NO: 3 - DNA, 1082 bp, Homo sapiens
  CDS (185)..(1012)
cagaatatcaatgctccatttattcttggtggaagataaagaaggctggctcctgttcctagcgccgaatgtgctcagaagcgtataggctgaagaagcgggcagtgataaattggctcatgagagtgtgtgaggaggttcagagcccatggggctcttggcctgaagtttcttgatcaagaaaatggaacctctcccaggatgggggtcggcaccctggagccaggcccctacagacaacaccttcaggctggtgccttgtgtcttccttttgaacttgctgcagcgcatctctgcccagccctcatgcagacaggaggagttccttgtgggagacgagtgctgccccatgtgcaacccaggttaccatgtgaagcaggtctgcagtgagcatacaggcacagtgtgtgccccctgtcccccacagacatataccgcccatgcaaatggcctgagcaagtgtctgccctgcggagtccgtgatccagacatgggcctgctgacctggcaggagtgctccagctggaaggacactgtgtgcagatgcatcccaggctacttctgtgagaaccaggatgggagccactgttccacatgcttgcagcacaccacctgccctccagggcagagggtagagaagagagggactcacgaccaggacactgtatgtgctgactgcctaacagggaccttctcacttggagggactcaggaggaatgcctgccctggaccaactgcagtgcatttcaacaggaagtaagacgtgggaccaacagcacagacaccacctgctcctcccaggtcgtctactacgttgtgtccatccttttgccacttgtgatagtgggagttgggatagctggattcctcatctgcacgcgaagacacctgcacaccagctcagtggccaaggagctggagcctttccagcaggaacaacaggagaacaccatcaggtttccagtcaccgaggttgggtttgctgagaccgaggaggagacagcctccaactgaacaaattctgggtgacaagacaccgaggagacgttggtgggaagtttccatatgctattgggacatg

SEQ ID NO: 4 - protein, 276 aa, Homo sapiens
MEPLPGWGSAPWSQAPTDNTFRLVPCVFLLNLLQRISAQPSCRQEEFLVGDECCPMCNPGYHVKQVCSEHTGTVCAPCPPQTYTAHANGLSKCLPCGVRDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYYVVSILLPLVIVGVGIAGFLICTRRHLHTSSVAKELEPFQQEQQENTIRFPVTEVGFAETEEETASN

SEQ ID NO: 5 - DNA, 1169 bp, Homo sapiens
  CDS (49)..(771)
gaggttgaaggacccaggcgtgtcagccctgctccagagaccttgggcatggaggagagtgtcgtacggccctcagtgtttgtggtggatggacagaccgacatcccattcacgaggctgggacgaagccaccggagacagtcgtgcagtgtggcccgggtgggtctgggtctcttgctgttgctgatgggggctgggctggccgtccaaggctggttcctcctgcagctgcactggcgtctaggagagatggtcacccgcctgcctgacggacctgcaggctcctgggagcagctgatacaagagcgaaggtctcacgaggtcaacccagcagcgcatctcacaggggccaactccagcttgaccggcagcggggggccgctgttatgggagactcagctgggcctggccttcctgaggggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggaggaggtggtcgtccgtgtgctggatgaacgcctggttcgactgcgtgatggtacccggtcttacttcggggctttcatggtgtgaaggaaggagcgtggtgcattggacatgggtctgacacgtggagaactcagagggtgcctcaggggaaagaaaactcacgaagcagaggctgggcgtggtggctctcgcctgtaatcccagcactttgggaggccaaggcaggcggatcacctgaggtcaggagttcgagaccagcctggctaacatggcaaaaccccatctctactaaaaatacaaaaattagccggacgtggtggtgcctgcctgtaatccagctactcaggaggctgaggcaggataattttgcttaaacccgggaggcggaggttgcagtgagccgagatcacaccactgcactccaacctgggaaacgcagtgagactgtgcctcaaaaaaaaaaaaaaaaaaaaaaaaaaaaa

SEQ ID NO: 6 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMV

SEQ ID NO: 7 - DNA, 1894 bp, Murine sp.
  CDS (115)..(834)
tcgacccacgcgtccgagcccgagcgtgttgggcaattgtggtttcctccggaagaggaggaactcaggcttgccaaccctttccctgggcttcggagcctcagctgctctggcatggagagtgtggtacagccttcagtgtttgtggtggatggacagacggacatcccattcaggcggctggaacagaaccaccggagacggcgctgtggcactgtccaggtcagcctggccctggtgctgctgctaggtgctgggctggccactcagggctggtttctcctgagactgcatcaacgtcttggagacatagtagctcatctgccagatggaggcaaaggctcctgggagaagctgatacaagatcaacgatctcaccaggccaacccagcagcacatcttacaggagccaacgccagcttgataggtattggtggacctctgttatgggagacacgacttggcctggccttcttgaggggcttgacgtatcatgatggggccctggtgaccatggagcccggttactactatgtgtactccaaagtgcagctgagcggcgtgggctgcccccaggggctggccaatggcctccccatcacccatggactatacaagcgcacatcccgctacccgaaggagttagaactgctggtcagtcggcggtcaccctgtggccgggccaacagctcccgagtctggtgggacagcagcttcctgggcggcgtggtacatctggaggctggggaagaagtggtggtccgcgtgcctggaaaccgcctggtcagaccacgtgacggcaccaggtcctatttcggagctttcatggtctgaaggctgcggtgacaatgtattttgtggagggacctctccaggactcacctcaaacccagcaatagggtttgaagtcctccctttaagnnnncctgaactctgcagtgctcggggcggtgtagactgctgacctgctttgggcaatcttcaaatcagagacctggagacttggggcgtggagcccaggagcgagggtcagctcatttgcctgatattcaggaagaaagaatcaagctggggtatttatgcttctgatgcaacactgagatttcggctttctgggttttgagctggaggcaagaaccttcccagagtgtcatcaggaccatgttggcaggacttggggctccagacttgccaccacactctggcctctcccatccatccgctgcattggtttccagccaccaaaacagcactggccccctggctgcaactggccaggtacgagcttctgagcacctacattcctcagggacatcttgatgagatctcagtactcagtccaatgcgcagcagcgacagacatgccaggaatggttggtcagaagggaagggaggaaagggaggaaagaagggaatgcagaagagaaggggggaaaacaagaccaaaacaaaacagcaacaacaaagcggcagggaggaggtgacacccttggggatactttagtcaacacacttagaacagattgtgccaggcctgttggattcctggagttgatgggatcgtgggaaggcacaatggggagcaagtgggcttgggttatggctcagtgggtaaagtgcaattatggggatctgagtttgaatccctggtacccatataaagacacagatgcggtgatgggcacttgtgacaatgagatcatcaatagggaatggagacaggagggacctctggggttcactggccaggcagtctagctgaatcaaagagctccaagttcagtcgatagctcctgaagatgacaactgaggctattctccaaaccccacacgcaggacacatgcgtaataaataaaattttaaaaatattaaaaaaaaaaaaaaaaaaaaaaa

SEQ ID NO: 8 - protein, 239 aa, Murine sp.
MESVVQPSVFVVDGQTDIPFRRLEQNHRRRRCGTVQVSLALVLLLGAGLATQGWFLLRLHQRLGDIVAHLPDGGKGSWEKLIQDQRSHQANPAAHLTGANASLIGIGGPLLWETRLGLAFLRGLTYHDGALVTMEPGYYYVYSKVQLSGVGCPQGLANGLPITHGLYKRTSRYPKELELLVSRRSPCGRANSSRVWWDSSFLGGVVHLEAGEEVVVRVPGNRLVRPRDGTRSYFGAFMV

SEQ ID NO: 9 - protein, 193 aa, Homo sapiens [sequence omitted]

SEQ ID NO: 10 - protein, 277 aa, Homo sapiens
MEPPGDWGPPPWRSTPRTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQVLILLPLSLPPPPSHLPSPRWGWCFWCTWWGLPVL

SEQ ID NO: 11 - protein, 186 aa, Homo sapiens
MEPPGDWGPPPWRSTPRTDVSRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKKA

SEQ ID NO: 12 - DNA, 1206 bp, Herpes simplex virus
  CDS (1)..(1185)
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctgtccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacataccgtcgatccaggacgccgcgacgccttaccatcccccggccaccccgaacaacatgggcctgatcgccggcgcggtgggcggcagtctcctggcagccctggtcatttgcggaattgtgtactggatgcgccgccgcactcaaaaagccccaaagcgcatacgcctcccccacatccgggaagacgaccagccgtcctcgcaccagcccttgttttactagatacccccccttaatgggtgc

SEQ ID NO: 13 - protein, 394 aa, Herpes simplex virus
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMRRRTQKAPKRIRLPHIREDDQPSSHQPLFY

SEQ ID NO: 14 - DNA, 5033 bp, Homo sapiens
  CDS (2583)..(2681)
tattggccataacccttatgatgggtcaggaagggatcacccctttctgcccctacagaaataatagctaagactagtaaagcataaaaggcaaaggggcaggtcctcaagtagagaagaacaggagaaatagctcatacacacccagaatgttacttacatgtccctccatgttacaccaagacccctcagggaccttgtgcctggggagagaagtggtctgccccatgcaacagtgggctttaccccgggtcaccaccagccccagctccaacccctctaacactctccaagtaaaatcacatcagtagcagtaataatatttgaggtgacaagttggtattatctcaaacttaggaaaagtgaataaagtcatctttagaaactgctttttttaaaccttgtaacttgcaagctaagtgaaaatgggctcatgtatgagaatgttcgtgttagacattttttgtgttagacaaaaactagaaacaaaccaaatccccatcaacagaatatattagaatatattgatacaatagaatattacatcataattttttttaaaaacattactgatacatacaaccacgtatatgaatctcacaaacataatgctgactgaaagaagtcaaacagaaatgagtacattctgtgtgatttcatttatatgatgccccaaaccaggaggaaataatctatggtgataaaagtgagagagtggttggttatctttggagggtatcagcagggagggggcatgagggaacctgctggggacctgaaaatacgtggagctgggtggtggctacatacagatggaaaaattcatcagctgtacacttaagaggtgtccacctcatacctaagttacatatcaataaaaaggaaaaaaattttggaaacttttttttttttttttgagacagagtcttgctctgtcccccaggctggaatacagtggtgcgatcttgactcactgcagcctccgcctcccaggttcaaataattctccagcctcagcctcccgagtagctgggactgcagatgcgcaccagcacgcctggctaatttttgtatttattatagagatggggtttcaccatgttggccagctggtctcaaactcctgacctcaagtaatccgcccacctcagactcccaaagtgccaggattacaggtgtgagccactgcaccaggcctggaacaattttaaaataatgtattggctctgcaaatgcagcttcagaacaagtcccttagctgtccccaccccaccctaagtcaccacccttaagcctcacccatgtggaattctgaaacttcctttgtagaaaactttggaaggtgtctgccacattgatcctggaatgtgtgtttatttggggttatataaatctgttctgtggaagccacctgaagtcaggaagagatggagggcatccttcaggagtgagatgagacctcatcatacttgactgtccagcatcatctctgagtaaggggaccaaaaaatttatcttccaaactaggacactttcaagagtggaagggggatccattaatattttcacctggacaagaggcaaacaccagaatgtccccgatgaaggggatatataatggaccttcttgatgtgaaacctgccagatgggctggaaagtccgtatactgggacaagtatgatttgagttgtttgggacaaggacaggggtacaagagaaggaaatgggcaaagagagaagcctgtactcagccaagggtgcagagatgttatatatgattgctcttcagggaaccgggcctccagctcacaccccagctgctcaaccgcctcctctctgaattgactgtcccttctttggaactctaggcctgaccccactccctggccctcccagcccacgattcccctgacccgactccctttcccagaactcagtcgcctgaacccccagcctgtggttctctcctaggcctcagcctttcctgcctttgactgaaacagcagtatcttctaagccctgggggcttccccgggccccagccccgacctagaacccgcccgctgcctgccacgctgccactgccgcttcctctataaagggacctgagcgtccgggcccaggggctccgcacagcaggtgaggctctcctgccccatctccttgggctgcccgtgcttcgtgctttggactaccgccccgcagtgtcctgccctctgcctgggcctcggtccctcctgcacctgctgcctggatccccggcctgcctgggcctgggccttggtgggtttggttttggtttccttctctgtctctgactctccatctgtcagtctcattgtctctgtcacacattctctgtttctgccatgattcctctctgttcccttcctgtctctctctgtctccctctgctcaccttggggtttctctgactgcatcttgtccccttctctgtcgatctctctctcgggggtcggggggtgctgtctcccagggcgggaggtctgtcttccgccgcgtgccccgccccgctcactgtctctctctctctctctctctttctctgcaggttctccccatgacaccacctgaacgtctcttcctcccaagggtgtgtggcaccaccctacacctcctccttctggggctgctgctggttctgctgcctggggcccaggtgaggcagcaggagaatgggggctgctggggtggctcagccaaaccttgagccctagagcccccctcaactctgttctcccctaggggctccctggtgttggcctcacaccttcagctgcccagactgcccgtcagcaccccaagatgcatcttgcccacagcaccctcaaacctgctgctcacctcattggtaaacatccacctgacctcccagacatgtccccaccagctctcctcctacccctgcctcaggaacccaagcatccacccctctcccccaacttcccccacgctaaaaaaaacagagggagcccactcctatgcctccccctgccatcccccaggaactcagttgttcagtgcccacttcctcagggattgagacctctgatccagacccctgatctcccacccccatcccctatggctcttcctaggagaccccagcaagcagaactcactgctctggagagcaaacacggaccgtgccttcctccaggatggtttctccttgagcaacaattctctcctggtccccaccagtggcatctacttcgtctactcccaggtggtcttctctgggaaagcctactctcccaaggccacctcctccccactctacctggcccatgaggtccagctcttctcctcccagtaccccttccatgtgcctctcctcagctcccagaagatggtgtatccagggctgcaggaaccctggctgcactcgatgtaccacggggctgcgttccagctcacccagggagaccagctatccacccacacagatggcatcccccacctagtcctcagccctagtactgtcttctttggagccttcgctctgtagaacttggaaaaatccagaaagaaaaaataattgatttcaagaccttctccccattctgcctccattctgaccatttcaggggtcgtcaccacctctcctttggccattccaacagctcaagtcttccctgatcaagtcaccggagctttcaaagaaggaattctaggcatcccaggggaccacacctccctgaaccatccctgatgtctgtctggctgaggatttcaagcctgcctaggaattcccagcccaaagctgttggtctgtcccaccagctaggtggggcctagatccacacacagaggaagagcaggcacatggaggagcttgggggatgactagaggcagggaggggactatttatgaaggcaaaaaaattaaattatttatttatggaggatggagagaggggaataatagaagaacatccaaggagaaacagagacaggcccaagagatgaagagtgagagggcatgcgcacaaggctgaccaagagagaaagaagtaggcatgagggatcacagggccccagaaggcagggaaaggctctgaaagccagctgccgaccagagccccacacggaggcatctgcaccctcgatgaagcccaataaacctcttttctctgaaatgctgtctgcttgtgtgtgtgtgtctgggagtgagaacttcccagtctatctaaggaatggagggagggacagagggctcaaagggagcaagagctgtggggagaacaaaaggataagggctcagagagcttcagggatatgtgatggactcaccaggtgaggccgccagactgctgcaggggaagcaaaggagaagctgagaagatgaaggaaaagtcagggtctggaggggcgggggtcagggagctcctgggagatatggccacatgtagcggctctgaggaatgggttacaggagacctctggggagatgtgaccacagcaatgggtaggagaatgtccagggctatggaagtcgagtatggggacccccccttaacgaagacagggccatgtagagggccccagggagtgaaagagcctccaggacctccaggtatggaatacaggggacgtttaagaagatatggccacacactggggccctgagaagtgagagcttcatgaaaaaaatcagggaccccagagttccttggaagccaagactgaaaccagcattatgagtctccgggtcagaatgaaagaagaaggcctgccccagtggggtctgtgaattcccgggggtgatttcactccccggggctgtcccaggcttgtccctgctacccccacccagcctttcctgaggcctcaagcctgccaccaagcccccagctccttctccccgcagggacccaaacacaggcctcaggactcaacacagcttttccctccaaccccgttttctctccctcaaggactcagctttctgaagcccctcccagttctagttctatctttttcctgcatcctgtctggaagttagaag

SEQ ID NO: 15 - protein, 205 aa, Homo sapiens;
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 16 - DNA, 2105 bp, Homo sapiens
  CDS (169)..(1476)
gccctggaggcccggcctggccgctcccggccctggggtgcacatcggccctgagtcccgtcccaggctctgggctcgggcagccgccgccaccgctgcccaggacgtcgggcctcctgccttcctcccaggcccccacgttgctggccgcctggccgagtggccgccatgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactgactgagtctgagaaaaggcagaagaaggggggcacaagggcaccttctcccttgaggctgccctgcccacgtgggattcacaggggcctgagtagggcccggggaagcagagccctaagggattaaggctcagacacctctgagagcaggtgggcactggctgggtacggtgccctccacaggactctccctactgcctgagcaaacctgaggcctcccggcagacccacccaccccctggggctgctcagcctcaggcacggacagggcacatgataccaactgctgcccactacggcacgccgcaccggagcacggcaccgagggagccgccacacggtcacctgcaaggacgtcacgggcccctctaaaggattcgtggtgctcatccccaagcttcagagaccctttggggttccacacttcacgtggactgaggtagaccctgcatgaagatgaaattatagggaggacgctccttccctcccctcctagaggagaggaaagggagtcattaacaactagggggttgggtaggattcctaggtatggggaagagttttggaaggggaggaaaatggcaagtgtatttatattgtaaccacatgcaaataaaaagaatgggaccta

SEQ ID NO: 17 - protein, 435 aa, Homo sapiens
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 18 - DNA, 903 bp, Homo sapiens
  CDS (1)..(903)
atgagggcgctggaggggccaggcctgtcgctgctgtgcctggtgttggcgctgcctgccctgctgccggtgccggctgtacgcggagtggcagaaacacccacctacccctggcgggacgcagagacaggggagcggctggtgtgcgcccagtgccccccaggcacctttgtgcagcggccgtgccgccgagacagccccacgacgtgtggcccgtgtccaccgcgccactacacgcagttctggaactacctggagcgctgccgctactgcaacgtcctctgcggggagcgtgaggaggaggcacgggcttgccacgccacccacaaccgtgcctgccgctgccgcaccggcttcttcgcgcacgctggtttctgcttggagcacgcatcgtgtccacctggtgccggcgtgattgccccgggcacccccagccagaacacgcagtgccagccgtgccccccaggcaccttctcagccagcagctccagctcagagcagtgccagccccaccgcaactgcacggccctgggcctggccctcaatgtgccaggctcttcctcccatgacaccctgtgcaccagctgcactggcttccccctcagcaccagggtaccaggagctgaggagtgtgagcgtgccgtcatcgactttgtggctttccaggacatctccatcaagaggctgcagcggctgctgcaggccctcgaggccccggagggctggggtccgacaccaagggcgggccgcgcggccttgcagctgaagctgcgtcggcggctcacggagctcctgggggcgcaggacggggcgctgctggtgcggctgctgcaggcgctgcgcgtggccaggatgcccgggctggagcggagcgtccgtgagcgcttcctccctgtgcactga

SEQ ID NO: 19 - protein, 300 aa, Homo sapiens
MRALEGPGLSLLCLVLALPALLPVPAVRGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 20 - DNA, 23 bp, Artificial
  Description of Artificial Sequence Primer
atggagagtgtggtacagccttc

SEQ ID NO: 21 - DNA, 23 bp, Artificial
  Description of Artificial Sequence Primer
gaccatgaaagctccgaaatagg

SEQ ID NO: 22 - DNA, 22 bp, Artificial
  Description of Artificial Sequence Primer
atggaacctctcccaggatggg

SEQ ID NO: 23 - DNA, 22 bp, Artificial
  Description of Artificial Sequence Primer
tcagttggaggctgtctcctcc